SEQUENCE LISTING (46 sequences)

SEQ ID NO: 1 - DNA, 22 bp, Artificial Sequence
  Synthetic
ctccartatttgcyataaagaa

SEQ ID NO: 2 - DNA, 22 bp, Artificial Sequence
  Synthetic
ctccartatttgcyataaagag

SEQ ID NO: 3 - DNA, 23 bp, Artificial Sequence
  Synthetic
raargacagtactaagtggagaa

SEQ ID NO: 4 - DNA, 23 bp, Artificial Sequence
  Synthetic
aaaagayagyactaaatggagra

SEQ ID NO: 5 - DNA, 24 bp, Artificial Sequence
  Synthetic
catccagcrgggytaaaaaagaar

SEQ ID NO: 6 - DNA, 24 bp, Artificial Sequence
  Synthetic
catccagcrgggytaaaaaagaay

SEQ ID NO: 7 - DNA, 26 bp, Artificial Sequence
  Synthetic
aaatcagtracagtactrgatgtggg

SEQ ID NO: 8 - DNA, 28 bp, Artificial Sequence
  Synthetic
ccagcagggttaaaaaagaaaaaatcag

SEQ ID NO: 9 - DNA, 28 bp, Artificial Sequence
  Synthetic
ccagcagggttaaaaaagaaaaaatcaa

SEQ ID NO: 10 - DNA, 29 bp, Artificial Sequence
  Synthetic
tracagtactrgatgtgggggatgcatat

SEQ ID NO: 11 - DNA, 24 bp, Artificial Sequence
  Synthetic
aaaaaatccagaaatarttatyta

SEQ ID NO: 12 - DNA, 24 bp, Artificial Sequence
  Synthetic
aaaaaatccagaaatarttatytg

SEQ ID NO: 13 - DNA, 25 bp, Artificial Sequence
  Synthetic
ycaatacatggatgayttgtatgta

SEQ ID NO: 14 - DNA, 27 bp, Artificial Sequence
  Synthetic
atccagaaatarttatctatcaataya

SEQ ID NO: 15 - DNA, 27 bp, Artificial Sequence
  Synthetic
atccagaaatarttatctatcaatayg

SEQ ID NO: 16 - DNA, 25 bp, Artificial Sequence
  Synthetic
tggatgayttgtatgtaggatctga

SEQ ID NO: 17 - DNA, 21 bp, Artificial Sequence
  Synthetic
catggatgayttgtatgtrgg

SEQ ID NO: 18 - DNA, 21 bp, Artificial Sequence
  Synthetic
catggatgayttgtatgtrgc

SEQ ID NO: 19 - DNA, 24 bp, Artificial Sequence
  Synthetic
atctgayttagaaatagggcagca

SEQ ID NO: 20 - DNA, 23 bp, Artificial Sequence
  Synthetic
ttccctacaatccccaaagtcar

SEQ ID NO: 21 - DNA, 23 bp, Artificial Sequence
  Synthetic
ttccctacaatccccaaagtcac

SEQ ID NO: 22 - DNA, 25 bp, Artificial Sequence
  Synthetic
ggagtagtagaatcyatgaataaag

SEQ ID NO: 23 - DNA, 23 bp, Artificial Sequence
  Synthetic
attccctacaatccccaaagtca

SEQ ID NO: 24 - DNA, 23 bp, Artificial Sequence
  Synthetic
attccctacaatccccaaagtcg

SEQ ID NO: 25 - DNA, 24 bp, Artificial Sequence
  Synthetic
grattccctacaatccccaaagyc

SEQ ID NO: 26 - DNA, 24 bp, Artificial Sequence
  Synthetic
grattccctacaatccccaaagyc

SEQ ID NO: 27 - DNA, 24 bp, Artificial Sequence
  Synthetic
grattccctacaatccccaaagya

SEQ ID NO: 28 - DNA, 25 bp, Artificial Sequence
  Synthetic
arggagtagtagaatcyatgaataa

SEQ ID NO: 29 - DNA, 24 bp, Artificial Sequence
  Synthetic
gtcaaggagtagtagaatcyatra

SEQ ID NO: 30 - DNA, 24 bp, Artificial Sequence
  Synthetic
gtcaaggagtagtagaatcyatrc

SEQ ID NO: 31 - DNA, 27 bp, Artificial Sequence
  Synthetic
ayaaagaattaaagaaaatyataggrc

SEQ ID NO: 32 - DNA, 24 bp, Artificial Sequence
  Synthetic
tgacataaargtagtrccaagrag

SEQ ID NO: 33 - DNA, 24 bp, Artificial Sequence
  Synthetic
tgacataaargtagtrccaagraa

SEQ ID NO: 34 - DNA, 25 bp, Artificial Sequence
  Synthetic
raaagcaaaratcattagggattat

SEQ ID NO: 35 - DNA, 26 bp, Artificial Sequence
  Synthetic
raaagyaaaaatcattaaggactatg

SEQ ID NO: 36 - DNA, 22 bp, Artificial Sequence
  Synthetic
tcctccgcgggcatgaccagtc

SEQ ID NO: 37 - DNA, 22 bp, Artificial Sequence
  Synthetic
tcctccgcgggcatgaccagta

SEQ ID NO: 38 - DNA, 23 bp, Artificial Sequence
  Synthetic
ygcctacgacggcaaggattaca

SEQ ID NO: 39 - DNA, 24 bp, Artificial Sequence
  Synthetic
ctcggtgcctttagtgatggcctg

SEQ ID NO: 40 - DNA, 23 bp, Artificial Sequence
  Synthetic
gctcacctggacaacctcaaggg

SEQ ID NO: 41 - DNA, 34 bp, Artificial Sequence
  Synthetic
tagcaaaagaaatagtagcyagctgtgataaatg

SEQ ID NO: 42 - DNA, 28 bp, Artificial Sequence
  Synthetic
caatcakcacctgccatctgttttccat

SEQ ID NO: 43 - DNA, 23 bp, Artificial Sequence
  Synthetic
ccagggtctcacatcatccaggt

SEQ ID NO: 44 - DNA, 20 bp, Artificial Sequence
  Synthetic
cgcctcccacttgcgctggg

SEQ ID NO: 45 - DNA, 25 bp, Artificial Sequence
  Synthetic
gggatctgtccactcctgatgctgt

SEQ ID NO: 46 - DNA, 24 bp, Artificial Sequence
  Synthetic
atccacgtgcagcttgtcacagtg

(19) United States
(12) Patent Application Publication
(10) Pub. No.: US 2021/0207206 A1
(43) Pub. Date: Jul. 8, 2021

(54) SYSTEMS AND METHODS FOR LIGATION

(71) Applicants: UNIVERSITY OF WASHINGTON, Seattle, WA (US); SEATTLE CHILDREN'S RESEARCH INSTITUTE, Seattle, WA (US)

(72) Inventors: Lisa FRENKEL, Seattle, WA (US); Ingrid BECK, Seattle, WA (US); Barry LUTZ, Seattle, WA (US); Nuttada PANPRADIST, Seattle, WA (US)

(73) Assignees: University of Washington, Seattle, WA (US); Seattle Children's Research Institute, Seattle, WA (US)

(21) Appl. No.: 17/055,966

(22) PCT Filed: May 17, 2019
(86) PCT No.: PCT/US2019/033005

§ 371 Date: Nov. 16, 2020

Related U.S. Application Data
(60) Provisional application No. 62/672,882, filed on May 17, 2018.

(51) Int. Cl. C12Q 1/6855; C12Q 1/6827; C12Q 1/70

(52) U.S. Cl. CPC C12Q 1/6855; C12Q 1/703; C12Q 1/6827

(57) ABSTRACT

The present technology provides systems and methods for oligonucleotide ligation assays (“OLA”). In some embodiments, OLA devices are provided that incorporate pathogen detection testing and drug resistance testing into a single device using a lateral flow membrane. The OLA devices can be used at point of car settings to aid a clinician in selecting an appropriate therapeutic regimen for an infected patient.

### CROSS-REFERENCE TO RELATED APPLICATION(S)

The present application claims priority to U.S. Provisional Patent Application No. 62/672,882, titled “SYSTEM AND METHOD FOR LIGATION,” filed May 17, 2018, the disclosure of which is hereby incorporated by reference in its entirety.

### STATEMENT REGARDING FEDERALLY SPONSORED RESEARCH

This invention was made with government support under Grant Nos. AI027757 and R01 A1110375, awarded by the National Institutes of Health. The government has certain rights in the invention.

### APPENDICES

Appendix 1 filed herewith further describes certain embodiments of the present technology, and forms part of the original disclosure provided herein.

### TECHNICAL FIELD

The present technology relates generally systems, devices, and methods for pathogen detection and characterization. In particular, the present technology provides oligonucleotide ligation assay devices for detecting the presence of one or more mutants within a pathogen.

### BACKGROUND

Nearly 40 million people worldwide are HIV-infected. Effective antiretroviral therapy (ART) suppresses viral replication, which allows HIV-infected individuals to live healthy and productive lives and reduces their risk of transmitting HIV to others. However, the error-prone HIV reverse transcription generates single-base mutants that can confer HIV drug resistance (HIVDR). Mutants at specific locations correlate closely with in vitro susceptibility testing and with clinical treatment failure. Recently, the prevalence of HIV drug resistance (HIVDR) has increased globally and threatens to render some existing drugs ineffective. More specifically, HIVDR to non-nucleoside reverse transcriptase inhibitors (NRTI) used in first-line ART continues to increase and pose a threat to successfully treating and repressing HIV in infected person. For example, persons with transmitted HIVDR may fail to achieve suppression of HIV replication after ART initiation. Additionally, when individuals do not adhere to their daily ART regimen, virus replication may not be suppressed (i.e., treatment failure), and HIVDR variants may be selected.

### BRIEF DESCRIPTION OF THE DRAWINGS

FIG. 1 is an illustration of a workflow for performing an oligonucleotide ligation assay (“OLA”) using thermal cycling.

FIG. 2A is an illustration of a workflow for performing an OLA using asymmetrical amplification and substantially isothermal ligation in accordance with embodiments of the present technology.

FIG. 2B is an illustration of a system for capturing ligated probes on a lateral flow membrane in accordance with embodiments of the present technology.

FIG. 2C illustrates the results of a drug resistance test conducted using isothermal ligation on a lateral flow membrane in accordance with embodiments of the present technology.

FIG. 2D is a graph reporting signal intensity of the results of the drug resistance test of FIG. 2C.

FIG. 3A is a block diagram illustrating a method of detecting a single nucleotide polymorphism in a target strand of DNA or RNA in accordance with embodiments of the present technology.

FIG. 3B is a block diagram illustrating a method for identifying drug resistant HIV using asymmetrical amplification and substantially isothermal ligation in accordance with embodiments of the present technology.

FIGS. 4A-4N are block diagrams illustrating select orders of workflow to determine the pathogen load and drug resistance in a biological sample containing a target strand of DNA or RNA in accordance with embodiments of the present technology.

FIG. 5A is an isometric view of an OLA device for testing pathogen load and drug resistance of a biological sample in accordance with embodiments of the present technology.

FIGS. 5B and 5C are enlarged views of the pathogen load detection zone and the drug resistance detection zone, respectively, of the OLA device illustrated in FIG. 5A.

FIG. 6A is an exploded isometric view of the OLA device illustrated in FIG. 5A.

FIG. 6B illustrates an interior of the OLA device illustrated in FIG. 5A and depicts a reagent pad and a lateral flow membrane configured in accordance with embodiments of the present technology.

FIGS. 7A-7H illustrate an embodiment of an OLA device that enables a user to selectively control reagent delivery to a lateral flow membrane in accordance with embodiments of the present technology.

FIGS. 8A-8F illustrate a workflow of the OLA device of FIG. 5A to detect a pathogen load and drug resistance of a target strand of DNA or RNA in accordance with embodiments of the present technology.

FIG. 9A illustrates an OLA device configured in accordance with embodiments of the present technology.

FIG. 9B is an enlarged view of the reagent pads of the OLA device of FIG. 9A and illustrates sequential delivery of reagents in accordance with embodiments of the present technology.

FIGS. 10A-10C illustrate an integrated sample collector and preparation device in accordance with embodiments of the present technology.

FIG. 11 illustrates a sample collecting tube in accordance with embodiments of the present technology.

FIG. 12A illustrates a workflow for capturing target pathogens on a lateral flow membrane in accordance with embodiments of the present technology.

FIG. 12B is a graph validating a process of selectively retaining target pathogens on a lateral flow membrane in accordance with embodiments of the present technology.

FIG. 13A illustrates a workflow for capturing a target DNA strand on a lateral flow membrane in accordance with embodiments of the present technology.

FIG. 13B is an illustration showing amplification following capture on a lateral flow membrane in accordance with embodiments of the present technology.

FIG. 13C illustrates a workflow for capturing a target RNA strand on a lateral flow membrane in accordance with embodiments of the present technology.

FIG. 13D is an illustration showing amplification following capture on a lateral flow membrane in accordance with embodiments of the present technology.

FIGS. 14A-14C illustrate an embodiment for selectively retaining and amplifying target strands on a lateral flow membrane in accordance with the present technology.

FIGS. 15A-15D illustrate steps of a method for high-throughput dried reagent fabrication in accordance with embodiments of the present technology.

### DETAILED DESCRIPTION

The present technology provides systems and methods for template dependent ligation. For example, some embodiments of the present technology provide systems and methods for substantially isothermal template dependent ligation for use with an oligonucleotide ligation assay (“OLA”). As will be described in greater detail below, substantially isothermal template dependent ligation can be useful in point of care settings to quickly and accurately detect the presence of certain genetic mutants. For example, the ligation systems and methods disclosed herein can be used to detect the presence of single nucleotide polymorphisms in a target region of an HIV nucleotide sequence that confers HIVDR. By detecting the presence of single nucleotide polymorphisms, the systems and methods provided herein can at least in part provide information on whether a drug regimen given to an HIV infected patient is no longer effective due to drug resistance mutants within the HIV genetic information. The results of the template dependent ligation assays can be interpreted in view of other test results (e.g., viral load testing) to determine other information useful in developing a treatment regimen for an HIV infected patient. For example, the combination of drug resistance and viral load can be used to determine patient adherence to a therapeutic regimen.

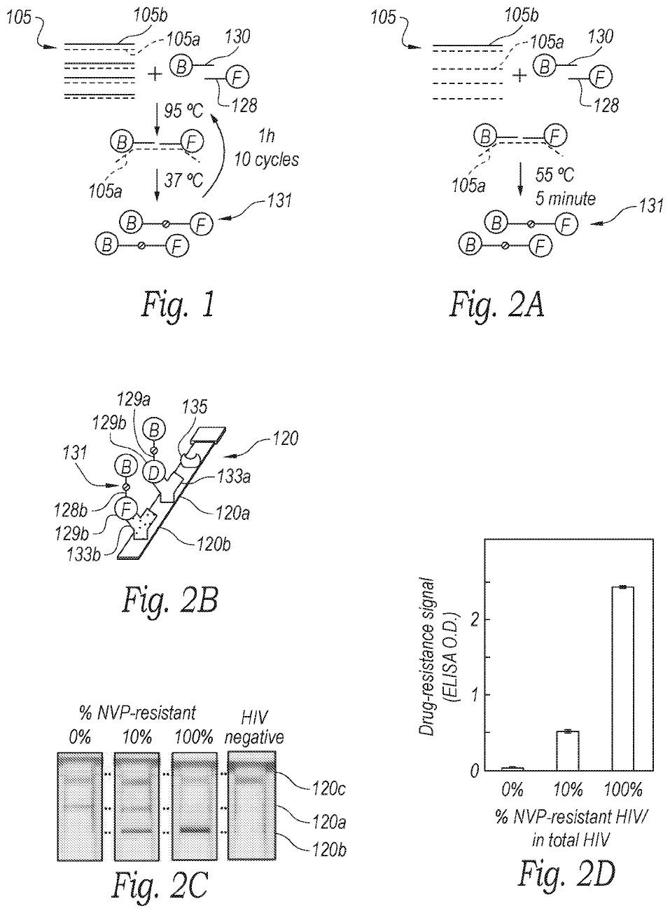

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the claims but are not described in detail with respect to FIGS. 1A-15D.

Reference throughout this specification to “one embodiment” or “an embodiment” means that a particular feature, structure or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “substantially,” “approximately,” and “about” are used herein to mean the stated value plus or minus 5%.

The headings provided herein are for convenience only and do not interpret the scope or meaning of the claimed present technology.

### Pathogen Load and Drug Resistance

Testing for HIVDR can identify the appropriate therapeutic option for an HIV-infected patient, including prevention of unnecessary switching of ART to more expensive regimens if treatment is failing due to low adherence rather than HIVDR. HIVDR testing can thus assist clinicians in selecting effective treatments. However, the complexity and cost of genotyping assays limit routine HIVDR testing in resource-limited settings where HIVDR prevalence has reached high levels. Commonly used Sanger sequencing is too expensive ($150-$300 per test) for routine HIVDR testing in many settings. Moreover, slow turnaround times for HIVDR genotypes are common due to the requirement of shipping specimens to centralized laboratories, thereby delaying changes in a therapeutic regimen for patients with a failing regimen.

Low aderence to medication can drastically impair drug efficiency and create opportunities for emerging drug-resistant pathogens. One approach to track how well a patient adheres to their medication is detection of byproducts or metabolites in urine or hair follicles. However, these metabolites may not be directly detectable by a rapid colorimetric method but rather require mass spec-based characterization methods and intensive data analysis. Current public health strategies rely on measuring viral or bacterial loads to indirectly evaluate if a person adheres to their medications. If the viral or bacterial load falls under a certain threshold (depending on the public health strategy), then that means that patient is taking their medications and has benefited from therapies. However, when the viral or bacterial loads are not suppressed, the clinicians provide repetitive counseling to ensure a patient is taking the medication and, if a high load persists, switch treatment. The multiple wait time points between tests can lead to loss to follow up and increased public health risks. This often leads to unnecessarily or delayed switching.

Certain embodiments of the present technology thus provide a synergistic system to gain the insights for an individual's adherence to a therapeutic regimen. For example, the combined use of a pathogen load test (e.g., a viral load and/or bacterial load test) and a drug resistance test can enable primary care clinicians to decide whether a patient needs to switch their treatment. The combined results of pathogen load and resistance testing can yield three potential outcomes that enable clinicians to identify if a person has adhered to their medication, displayed in Table 1.

TABLE 1
Potential outcomes from combined pathogen load and resistance testing

| | Drug Resistance | | |
|---|---|---|---|
| Pathogen load | test | Interpretation | Recommendation |
| Above | No detectable | Low adherence. | No need to switch treatment |
| threshold | drug resistance | Current drug should be | regimen, but focusing more |
| | | working fine. | on patient counseling |
| Above | Detectable drug | Current drug is no | Switch treatment regimen |
| threshold | resistance | longer effective. This | immediately |
| | | patient may not fit well | |
| | | with the current | |
| | | regimen or skipped | |
| | | doses which allow for | |
| | | pathogen to evolve and | |
| | | become resistant to | |
| | | current treatment | |
| Below | No detectable | Patient adheres to their | Encourage patient to |
| threshold | drug resistance | medication and the | continue to adhere to their |
| | | drug is working well. | treatment. No need to |
| | | | switch treatment regimen |

The present disclosure thus offers synergistic use of pathogen load and drug resistance test results to determine whether a patient adheres to their medication. These two tests can be run independently, simultaneously, and/or sequentially within a certain time frame. For example, the tests can be performed substantially simultaneously such that the test will provide results within a single patient visit (e.g., within 4 hours or less, within 3 hours or less, within 2 hours or less, within 1 hour or less, etc.). In some embodiments, the pathogen load test and the drug resistance test can be integrated into a single OLA device.

### Oligonucleotide Ligation Assays

Oligonucleotide ligation assays can identify a specific nucleotide sequence in a target strand without the use of radiochemicals, electrophoresis, or centrifugation. In its simplest form, OLAs use at least two probes: a test probe and a common probe. The test probe hybridizes to a first region of the target strand having a sequence complimentary to the test probe, and the common probe hybridizes to a second region of the target strand having a sequence complimentary to the common probe. The first and second regions can be immediately adjacent, or they can be spaced apart by one or more nucleotides. If the probes match the template (e.g., fully complimentary in a region surrounding the portion of the probes to be ligated), a ligase can join the two probes together in a process called ligation. The ligated probes can then be captured and/or detected to signal the presence of a specific nucleotide sequence.

OLAs can be used to detect any potential sequence on a target DNA or RNA strand. To test for a specific sequence, a test probe having a sequence corresponding to the specific sequence can be used. Certain embodiments of the present technology can include first test probes having a first nucleotide sequence comprising a first potential sequence of the target strand, second test probes having a second nucleotide sequence comprising a second potential sequence of the template strand, third test probes having a third nucleotide sequence comprising a third potential sequence of the template strand, etc. While the foregoing description discusses three test probes, the number of probes used can vary based on the number of potential sequences to be detected/tested for. In some embodiments, the first probe may have sequence corresponding to a wild type sequence, the second probe may have a sequence corresponding to a first mutant sequence (e.g., differing from the wild type sequence by a single nucleotide polymorphism), and the third probe may have a sequence corresponding to a second mutant sequence (e.g., differing from the wild type sequence by a single nucleotide polymorphism at a different position than the single nucleotide polymorphism of the first mutant sequence). In other embodiments, the first, second, and third probes have sequences corresponding to first, second, and third mutants, respectively.

As will be described in greater detail herein, the test probes can further include capture molecules to facilitate retention of ligated probes. For example, the first test probe may include a first hapten, a first immuno-tag, a first nucleotide barcode (e.g., a first unique DNA. PNA, LNA, or pDNA sequence), or other first molecule for selectively retaining the first test probe. The second test probe can include a second hapten, a second immuno-tag, a second nucleotide barcode (e.g., a second unique DNA, PNA, LNA, or pDNA sequence), or other second molecule for selectively retaining the second test probe. The third test probe may include a third hapten, a third immune-tag, a third nucleotide barcode (e.g., a third unique DNA, PNA, LNA, or pDNA sequence), or other third molecule for selectively retaining the third test probe. In some embodiments, the first, second, and third capture molecules are different.

The OLAs can also include common probes. The common probes can have a nucleic acid sequence substantially complimentary or complimentary to a second region of the template strand adjacent to the first region. Thus, when a common probe and a test probe are hybridized to the same template strand, the common probe and the test probe are adjacent to one another, and ligase (e.g., Taq DNA ligase) can ligate the common probe and the test probe by catalyzing the formation of a phosphodiester bond between a first end region of a test probe (e.g., the 5′ end of the test probe) and a first end region of the common probe (e.g., the phosphorylated 3′ end of the common end). Notably, while the first, second, and third test probes may compete to hybridize to the first region, the first, second, and third test probes will only be ligated to the common probe if their sequence is complimentary to the sequence of the template strand along the end region near the end that will be ligated to the common probe. In some embodiments, the test probe and the common probe can be immediately adjacent to another when hybridized to the template strand. In some embodiments, the test probe and the common probe can be spaced apart by one or more nucleotides when hybridized to the template strand. In some embodiments, the common probes can include a reporter molecule for providing a signal. For example, the common probes may include fluorescein, biotin, a hapten, or another molecule that can provide a detectable signal. In other embodiments, the common probes do not include reporter molecules.

A number of different ligated probes can form between the plurality of test probes and the common probes. For example, the ligated probes can include first ligated probes comprising the first test probe ligated to the common probe, second ligated probes comprising the second test probe ligated to the common probe, third ligated probes comprising the third test probe ligated to the common probe, etc. As will be discussed in more detail below, the first, second, and third ligated probes can be selectively captured and/or detected in first, second, and third regions of a detection zone to identify and quantify the presence of specific nucleic acid sequences in the target strand.

As one skilled in the art will appreciate, the probes (e.g., the wild type probes, the mutant probes, and/or the common probes) may not be perfectly complementary to the full region of the template strand to which they correspond. For example, certain target pathogens (e.g., HIV RNA) are highly variable. Accordingly, the probes can be made longer than required for ligation purposes and/or the assay can be run significantly below the probe melting temperature so that the probes can bind the template strand despite a few mismatches. Nonetheless, the OLA assays are still able to identify specific mutations (e.g., single nucleotide polymorphisms) due to the specificity of the ligase, which requires the bases near the ligation site to be perfect matches. For example, when the 5′ end of the test probe is to be ligated to the 3′ end of the common probe, ligation will only occur if the bases near the 5′ end region of the test probe and the 3′ end region of the common probe are perfectly complementary to the template strand. Likewise, when the 3′ end of the test probe is to be ligated to the 5′ end of the common probe, ligation will only occur if the bases near the 3′ end region of the test probe and the 5′ end region of the common probe are perfectly complementary to the template strand. Accordingly, the specific sequence of interest (e.g., the single nucleotide polymorphism) can be at or near the end of the test strand that will be ligated to the common strand to enable precise ligation and detection of specific sequences. For example, when the 5′ end of the test strand is to be ligated to the 3′ end of the common strand, the 5′ end region of the test strand can have the nucleotide corresponding to the single nucleotide polymorphism.

As one skilled in the art will appreciate, OLAs can be used in a variety of applications. The probe sequences will be selected based on the specific sequences the target strand is being tested for. When testing for HIVDR, for example, an OLA assay may test for a number of different mutations, including, for example, K65R, K103N, V106M, Y181C, M184V, G190A, Q148H, Q148R, Q148K, N155H. and/or R263K. Exemplary probes used to detect these mutant sequences, as well as associated wild type probes and common probes, are provided as SEQ. ID NOs. 1-35 in Table 2.

TABLE 2
Probes for HIV Drug Resistant Testing

| Codon | Mutation | Type | Sequence (5′→3′) | SEQ ID |
|---|---|---|---|---|
| 65 | K65R | WT: | CTCCARTATTTGCYATAAAGAA | 1 |
| | | MUT: | CTCCARTATTTGCYATAAAGAG | 2 |
| | | COM1: | RAARGACAGTACTAAGTGGAGAA | 3 |
| | | COM2: | AAAAGAYAGYACTAAATGGAGRA | 4 |
| 103 | K103N | WT: | CATCCAGCRGGGYTAAAAAAGAAR | 5 |
| | | MUT: | CATCCAGCRGGGYTAAAAAAGAAY | 6 |
| | | COM: | AAATCAGTRACAGTACTRGATGTGGG | 7 |
| 106 | V06M | WT: | CCAGCAGGGTTAAAAAAGAAAAAATCAG | 8 |
| | | MUT: | CCAGCAGGGTTAAAAAAGAAAAAATCAA | 9 |
| | | COM: | TRACAGTACTRGATGTGGGGGATGCATAT | 10 |
| 181 | Y181C | WT: | AAAAAATCCAGAAATARTTATYTA | 11 |
| | | MUT: | AAAAAATCCAGAAATARTTATYTG | 12 |
| | | COM: | YCAATACATGGATGAYTTGTATGTA | 13 |
| 184 | M184V: | WT: | ATCCAGAAATARTTATCTATCAATAYA | 14 |
| | | MUT: | ATCCAGAAATARTTATCTATCAATAYG | 15 |
| | | COM: | TGGTGAYTTGTATGTAGGATCTGA | 16 |
| 190 | G190A | WT: | CATGGATGAYTTGTATGTRGG | 17 |
| | | MUT: | CATGGATGAYTTGTATGTRGC | 18 |
| | | COM: | ATCTGAYTTAGAAATAGGGCAGCA | 19 |
| 148 | Q148H | WT: | TTCCCTACAATCCCCAAAGTCAR | 20 |
| | | MUT: | TTCCCTACAATCCCCAAAGTCAC | 21 |
| | | COM: | GGAGTAGTAGAATCYATGAATAAAG | 22 |
| 148 | Q148R | WT: | ATTCCCTACAATCCCCAAAGTCA | 23 |
| | | MUT: | ATTCCCTACAATCCCCAAAGTCG | 24 |
| | | COM: | RGGAGTAGTAGAATCYATGAATAAAG | 25 |
| 148 | Q148K | WT: | GRATTCCCTACAATCCCCAAAGYC | 26 |
| | | MUT: | GRATTCCCTACAATCCCCAAAGYA | 27 |
| | | COM: | ARGGAGTAGTAGAATCYATGAATAA | 28 |
| 155 | N155H | WT: | GTCAAGGAGTAGTAGAATCYATRA | 29 |
| | | MUT: | GTCAAGGAGTAGTAGAATCYATRC | 30 |
| | | COM: | AYAAAGAATTAAAGAAAATYATAGGRC | 31 |
| 263 | R263K | WT: | TGACATAAARGTAGTRCCAAGRAG | 32 |
| | | MUT: | TGCATAAARGTAGTRCCAAGRAA | 33 |
| | | COM1: | RAAGCAAARATCATTAGGGATTAT | 34 |
| | | COM2: | RAAAGYAAAAATCATTAAGGACTATG | 35 |

The present technology can also be used to test for the presence of certain human leukocyte antigen (HLA) genotypes. Certain HLA genotypes (e.g., HLA b57) indicate a patient may have a hypersensitivity to certain anti-retroviral therapies (e.g., Abacavir). Exemplary probes for HLA genotyping are provided as SEQ ID NOs. 36-38 in Table 3.

TABLE 3
Probes for HLA Genotyping

| Probe Name | Type | Sequence (5′→3′) | SEQ ID |
|---|---|---|---|
| B5701 | WT: | TCCTCCGCGGGCATGACCAGTC | 36 |
| B57xx | MUT: | TCCTCCGCGGGCATGACCAGTA | 37 |
| B57 common | COM: | YGCCTACGACGGCAAGGATTACA | 38 |

In certain embodiments of the present technology, human betoglobin can be used as a control for various OLA tests. Sequences for human betoglobin probes are provided as SEQ. ID. NOs. 39 and 40 in Table 4.

TABLE 4
Probes for Betoglobin Control

| Probe Name | Type | Sequence (5′→3′) | SEQ ID |
|---|---|---|---|
| Beta-Dig | WT | CTCGGTGCCTTTAGTGATGGCCTG | 39 |
| Beta-common | COM | GCTCACCTGGACAACCTCAAGGG | 40 |

As will be discussed in greater detail herein. OLA typically requires target strand of RNA or DNA to be amplified prior to the ligation assay. Accordingly, the present technology also provides primers corresponding to select regions of interest on target strands. For example, SEQ ID. NOs. 41-46 set forth sequences for primers used to amplify the HIV-integrase region, the HLA B57 region, and human betoglobin, as provided in Table 5.

TABLE 5
Primers

| | Name | Type | Sequence (5′→3′) | SEQ ID |
|---|---|---|---|---|
| HIV-2 | Mod 2.5 | Forward | TAGCAAAAGAAATAGTAGCYAGCTGT | 41 |
| Integrase | INT | | GATAAATG | |
| | INT R2 | Reverse | CAATCAKCACCTGCCATCTGTTTTCCA | 42 |
| | | | T | |
| HLA B57 | B57 F | Forward | CCAGGGTCTCACATCATCCAGGT | 43 |
| | B57 4R | Reverse | CGCCTCCCACTTGCGCTGGG | 44 |
| Human | BetaFOext | Forward | GGGATCTGTCCACTCCTGATGCTGT | 45 |
| Betoglobin | BetaROext | Reverse | ATCCACGTGCAGCTTGTCACAGTG | 46 |

The specific mutations, probes, and primers are provided herein as exemplary applications of the present technology. As one skilled in the art will appreciate from the disclosure, the present technology provides systems and methods that can test for a wide variety of mutations in various applications. Accordingly, the present technology is not limited to testing for the specific mutations or using the specific probes or primers disclosed herein.

### Template Dependent Ligation to Detect Specific Nucleotide Sequences

Certain embodiments of the present technology provide systems, devices, and methods for template dependent ligation to identify specific nucleic acid sequences in a region of interest on a target strand of DNA or RNA. For example, template dependent ligation can be used to identify the presence or absence of single nucleotide polymorphisms in a region of interest in a target strand. In certain embodiments, for example, template dependent ligation can be used to detect mutants within HIV genetic information.

FIG. 1 illustrates an exemplary workflow for template-dependent ligation relying on thermal cycling to determine whether a target strand of DNA or RNA from a biological sample contains a specific nucleic acid sequence (e.g., a wild type sequence or a mutant sequence). The target strand (not shown) can be amplified to produce DNA amplicons 105 using traditional amplification methods (e.g., via polymerase chain reaction). It is typically necessary to amplify the target strand to ensure that a sufficient quantity is available to enable template-dependent ligation. The DNA amplicons 105 can be double-stranded DNA amplicons comprising “template” strands 105a and “non-template” strands 105b. However, to perform template dependent ligation following amplification of the target strand, the double-stranded DNA amplicons 105 must be denatured such that the template strands 105a and the non-template strands 105b dissociate from one other to form single-stranded DNA. To facilitate dissociation, the double-stranded DNA amplicons 105 are traditionally heated to a temperature of about 95 degrees Celsius.

Either before or after the heating step, one or more probes (including test probes 128 and common probes 130) can be mixed with the DNA amplicons 105. The temperature can be reduced to a temperature that facilitates hybridization between the probes 128, 130 and the template strand 105a (e g., between about 35-45 degrees Celsius). As will be discussed in more detail herein, the probes 128, 130 hybridized to adjacent regions on the template strand 105a can be ligated and captured to detect the presence of single nucleotide polymorphisms. However, while the temperature decrease can enable the template strands 105a to anneal to the probes 128,130, a portion of the template strands 105a will re-hybridize to the non-template strand 105b rather than anneal to the probes 128, 130. Thus, there is a binding competition between the non-template strand 105b and the probes 128,130, resulting in a low ligation efficiency (about 2%). As a result, this traditional process is inefficient at producing sufficient hybridization between the template strand 105a and the probes 728,730, and thus is inefficient at producing ligated probes 731. To account for this inefficiency, the steps of heating and cooling are repeated in a process called thermal cycling. The number of cycles can vary on the quantity of ligated probes 131 needed, but a typical assay will require at least 10 cycles. After a sufficient amount of ligated probes have been formed, the ligated probes can be captured and detected through a variety of techniques. For example, the ligated probes can be detected through techniques known in the art such as enzyme-linked immunosorbent assays (ELISA).

As discussed above, the double-stranded DNA amplicons can be heated to dissociate the template strand and non-template strand to facilitate subsequent template dependent ligation. However, because the dissociated non-template strand competes with the probes for annealing to the template strand, the process of heating the amplified sample to promote dissociation and cooling the amplified sample to promote hybridization and ligation must be continually repeated in a process known as thermal cycling Certain embodiments of the present technology provide systems and methods for template dependent ligation that can negate the need for thermal cycling and therefore increase the efficiency of template dependent ligation.

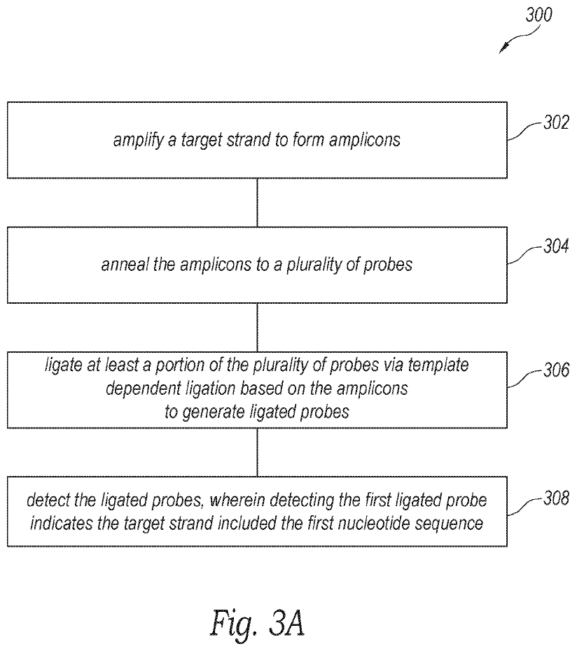

FIG. 2A illustrates an exemplary workflow of template dependent ligation according to select embodiments of the present technology. As illustrated, the template strand 105a can be provided in single strand form. In some embodiments, to provide the template strand 105a in single stranded from, the template strand 105a can be amplified at a higher rate than the non-template strand 105b, thereby producing excess single stranded amplicon. In some embodiments, the target strand can be amplified according to known techniques and, following amplification, treated to provide the amplicons in single-stranded form. Regardless of the technique used, there is a greater quantity and/or higher concentration of the template strand 105a than the non-template strand 105b following amplification. Because there is a greater quantity and/or higher concentration of the template strand 105a, the sample does not need to be heated to dissociate the double-stranded DNA amplicons 105. Thus, in some embodiments, certain techniques provided herein can eliminate the need to heat the sample to 95 degrees Celsius to free the template strand 105a from the non-template strand 105b to ready the sample for ligation. Rather, following amplification, the template strand 105a can be available in single-stranded form, and probes 128,130 can be added and bind to complimentary sequences along the template strand 105a.

Increasing the quantity and/or the concentration of the template strand to provide the template strand in single stranded form for template dependent ligation can be achieved through a variety of techniques. Such techniques can include pre-amplification techniques, amplification techniques, and post-amplification techniques (collectively referred to as “pre-ligation techniques”). In some embodiments, the pre-ligation techniques can be used after polymerase chain reaction or other amplification methods. As one of skill in the art will appreciate from the disclosure herein, these techniques can be used alone or in combination, and are used to prepare a sample for template dependent ligation.

Pre-amplification techniques involve treating the biological sample before amplification such that, following amplification, there is an increased concentration of the template strand versus the non-template strand. Pre-amplification techniques include treating the pre-amplified sample with an enzyme to digest one strand of the pre-amplified double-stranded DNA. For example, enzymes such as lambda exonuclease can selectively digest one strand of the double-stranded DNA (e.g., the phosphorylated 5′ end of a modified amplicon) to generate single stranded DNA template prior to amplification.

Amplification techniques include providing a first primer (e.g., a reverse primer) for amplifying a first strand at a higher concentration than a second primer (e.g., a forward primer) for amplifying a second strand to facilitate asymmetrical amplification. Using a first primer at a higher concentration than a second primer can generate partially single stranded DNA. As a result, after amplification, there is no need for heat treatment to dissociate the double-stranded DNA because there is sufficient single-stranded DNA. For example, in some embodiments, the primer for amplifying the template strand may be provided in higher concentrations than the primer for amplifying the non-template strand such that the template strand is provided in singe-stranded form following amplification.

Post-amplification techniques include techniques for generating single-stranded DNA amplicons after amplification of the target strand. For example, double-stranded DNA amplicons may be treated via one or more of the following techniques: (1) a heating method to induce strand breathing; (2) a strand displacement and exchange method; (3) enzymatic mediated strand invading; (4) enzymatic strand digestion; or (5) chemical dissociation methods. In the strand breathing technique, the double-stranded DNA amplicons can be incubated with the probes at temperatures about 5-10 degrees lower or higher than the melting temperature of the probes. While this temperature may not cause the double-stranded DNA to fully dissociate into single-stranded form, the increased temperature can cause the strands to partially dissociate in a process known as strand breathing. This slight dissociation can enable the probes to compete with the non-template strand for hybridization with the template strand. In the strand displacement and exchange technique, a single-stranded competing oligonucleotide can be mixed with the amplicons. If the single-stranded competing oligonucleotide forms a more thermodynamically stable double-stranded DNA duplex with the non-template strand, the template strand will dissociate from the non-template strand in favor of the more thermodynamically stable double-stranded DNA duplex. In the enzymatic mediated strand invading technique, recombinase enzymes can bind to single-stranded DNA or free single stranded probe and insert the probe into the duplex of the DNA template. In another example of enzymatic mediated strand invading, enzymes such as heat liable and/or heat stable helicase can be used to de-hybridize the double-stranded DNA and allow for the kinetically favorable probes to bind the de-hybridized template and get ligated. In enzymatic strand digestion, an enzyme that selectively digests one strand of the double-stranded DNA can be added to the sample. For example, lambda exonuclease can selectively digest one strand of the double-stranded DNA (e.g., the phosphorylated 5′ end of a modified amplicon) to provide the template strand in single stranded form. In chemical dissociation methods, the environment including the amplicons can be treated to facilitate dissociation between the double-stranded amplicons. For example, a strong base (e.g., sodium hydroxide) can be added. In other embodiments, a salt concentration of the environment can be reduced.

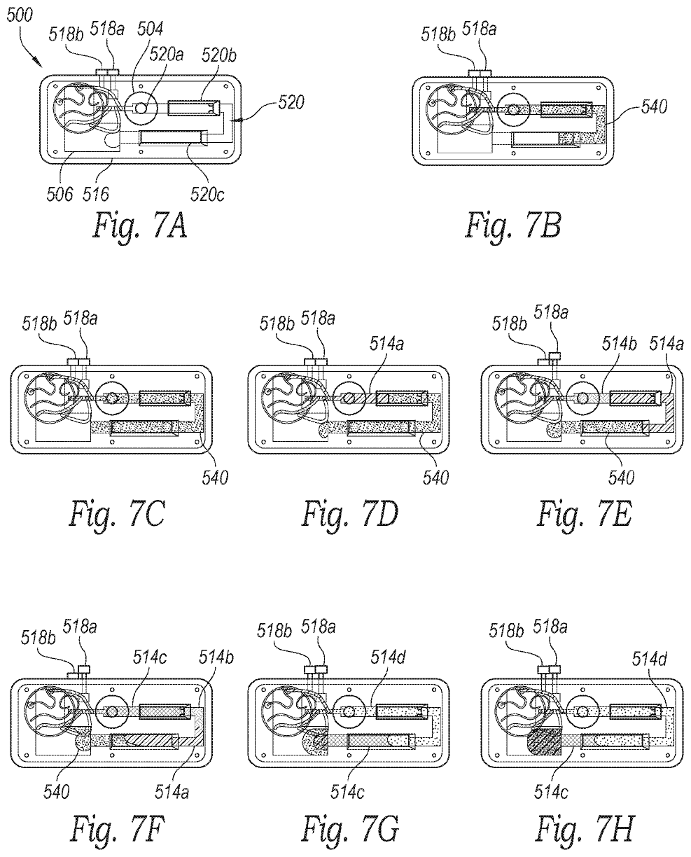

FIG. 2B illustrates a method of capturing the ligated probes 131 on a lateral flow membrane 120. More specifically, FIG. 2B illustrates using an immuno-tag to capture the ligated probes in distinct regions according to their sequence. For example, the first test probe 128a can include a first immuno-tag 129a and the second test probe 128b can include a second immuno-tag 129b. The first and second immuno-tags 129a, 129b can be different. Antibodies 133a corresponding to the first immuno-tag 129a can be immobilized in a first region 120a of a lateral flow membrane 120 and antibodies 133b corresponding to the second immuno-tag 129b can be immobilized in a second region 120b of the lateral flow membrane 120. As the ligated probes 131 flow through the lateral flow membrane 120, the antibodies 133 will bind to the immuno-common probes. Examples of immuno-tag/antibody pairs include FITC/anti-FITC and digoxigenin/anti-Dig, although other pairs known in the art can be used. As will be discussed in detail below with respect to FIGS. 8A-8F other capture mechanisms, such as computationally-designed DNA barcodes, can be used. Regardless of the capture mechanism used, the captured ligated probes 131 can be detected via a reporter molecule on the common probe 130. For example, the reporter molecule could include biotin, and the ligated probes 131 could be displayed through the reaction of streptavidin/alkaline phosphatase/substrate to create purple bands. In some embodiments, the lateral flow membrane 120 can include a biotin-BSA for flow control. In other embodiments, the common probes do not include a reporter molecule, and the ligated probes are instead distinguished from non-ligated probes by fragment analysis such as gel electrophoresis or capillary electrophoresis.

FIG. 2C illustrates the results of a drug resistance test conducted using isothermal ligation on a lateral flow membrane. Four separate biological samples were tested: HIV negative, 0% Nevirapine (NVP)-resistant HV, 10% NVP-resistant HIV, and 100% NVP-resistant HIV. The lateral flow membrane had capture molecules immobilized to capture ligated probes corresponding to the wild-type HIV at a first region 120a and to capture ligated probes corresponding to the NVP-resistant HIV at a second region 120b. A third region, 120c, can correspond to the biotin-BSA flow control. As illustrated, bands of color appear at specific regions to indicate the presence of specific mutants. The signal intensity can be used to determine the percentage of the target strands having the specific mutant. For example, the signal intensity in the second region 120b illustrates a difference in intensity between 0%, 10%, and 100% NVP-resistant HIV. This signal intensity can be quantified to facilitate selection of an appropriate treatment regimen. FIG. 2D is a graph reporting signal intensity for the 0%, 10%, and 100% NVP-resistant HIV tested in FIG. 2C. As illustrated, after five-minute incubation at 55 degrees Celsius, the resistance signal from 10% Nevirapine-resistant HIV is significantly different from 0% Nevirapine-resistant HIV. Thus, the present technology provides systems and methods for identifying samples with as low as 10% resistance.

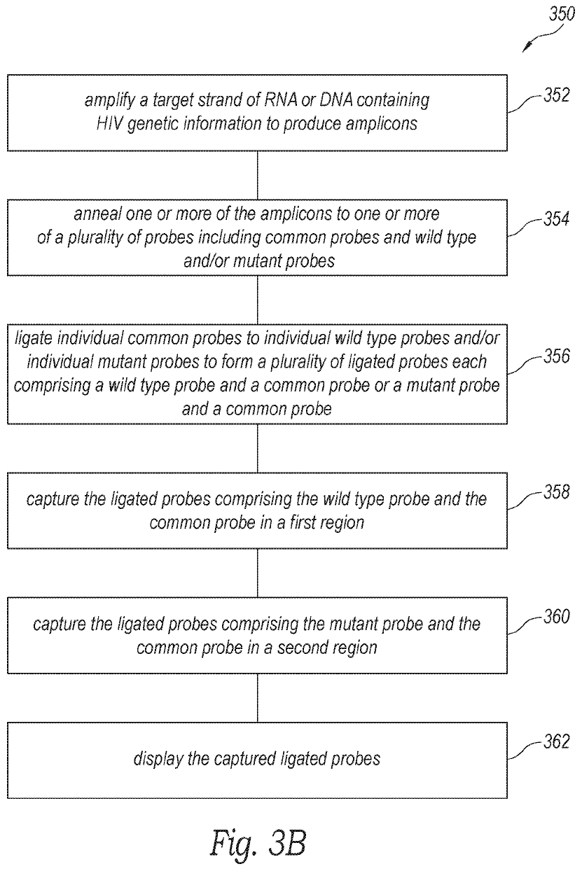

FIG. 3A illustrates a method 300 of detecting a single nucleotide polymorphism in a target strand. Method 300 can begin by amplifying a target strand to form amplicons (process step 302). One or more of the pre-ligation techniques discussed above can be used to provide a template strand in single stranded form. For example, asymmetrical amplification can be used and can result in (1) double-stranded amplicons comprising a template strand hybridized to a non-template strand, and (2) single stranded amplicons comprising the template strand. Because the target strand was asymmetrically amplified, the process does not require heating the amplified sample to dissociate the double-stranded amplicons. In other embodiments, the double-stranded amplicons are treated with other pre- or post-amplification techniques to provide the template strand in single stranded form.

Following amplification, probes can be combined with the amplified sample, and the single stranded amplicons can anneal to the probes (process step 304). The probes can include a first test probe having a first nucleotide sequence and a common probe having a second nucleotide sequence. The method 300 can continue by ligating at least a portion of the probes via template dependent ligation based on the single stranded amplicons to generate ligated probes (process step 306). The ligated probes can comprise first ligated probes having the first test probe ligated to the common probe. Different or constant probe concentrations can be used to perform ligation on single-stranded amplicons. The method 300 continues by detecting the ligated probes, wherein detecting the first ligated probe indicates the target strand included the first nucleotide sequence (process step 308). As noted above, the method 300 does not require thermal cycling, and can proceed to the step of capturing and/or detecting the ligated probes without the need for repeated cycles of heating and cooling. In some embodiments, the steps of asymmetrically amplifying, annealing, and ligating can be performed at temperatures at or below 70 degrees Celsius. In some embodiments, the steps of asymmetrically amplifying, annealing, and ligating can be performed at temperatures between 35-45 degrees Celsius. In some embodiments the step of asymmetrically amplifying is performed at 37 degrees Celsius and the step of ligating is performed at 40 degrees Celsius. In some embodiments, the steps of asymmetrically amplifying, annealing, and ligating are performed in a substantially isothermal environment. In some embodiments, the temperature is determined by the operating temperature range of the ligase (e.g., between about 37-65 degrees Celsius for Taq DNA Ligase).

Method 300 can further include second test probes for identifying second nucleotide sequences in the target strand. For example, the second test probes can have a second nucleotide sequence corresponding to a second potential sequence of the target strand. The second test probe can be ligated to the common probe if the second nuclotide sequence is present on the target strand. Accordingly, the second ligated probes can be detected to determine whether the target strand includes the second nucleotide sequence. As one skilled in the art will appreciate from the disclosure herein, additional test probes corresponding to additional potential sequences of the target strand can be added to determine whether the target strand has the potential sequence.

FIG. 3B illustrates a method 350 for identifying drug resistant HIV using asymmetrical amplification and substantially isothermal ligation. The method 350 can begin by amplifying a target strand of RNA or DNA containing HIV genetic information to produce amplicons (process step 352). As with method 300, one or more of the pre-ligation techniques discussed herein can be used to provide a template strand in single stranded form. For example, asymmetrical amplification can be used and can result in (1) double-stranded amplicons comprising a template strand hybridized to a non-template strand, and (2) single stranded amplicons comprising the template strand. Because the target strand was asymmetrically amplified, the process does not require heating the amplified sample to dissociate the double-stranded amplicons. In other embodiments, the double-stranded amplicons are treated with other pre- or post-amplification techniques to provide the template strand in single stranded form.

The method 350 can continue by annealing one or more of the single stranded amplicons to one or more of a plurality of probes (process step 354). The plurality of probes can include (1) wild type probes having a nucleotide sequence comprising a wild type sequence of the target strand, (2) at least one mutant probe having a nucleotide sequence comprising at least one potential mutant sequence of the target strand, the at least one potential mutant sequence including a single nucleotide polymorphism compared to the wild type sequence, and (3) common probes. In some embodiments, the plurality of probes could include additional mutant probes for testing other potential mutant sequences. In some embodiments, the method 350 can be performed without the wild type probe (i.e., using only mutant probes and common probes). The method 350 continues by ligating individual common probes to individual wild type probes and/or individual mutated probes to form a plurality of ligated probes each comprising a wild type probe and a common probe or a mutant probe and a common probe (process step 356). Ligation can occur when a wild type probe or a mutant probe is hybridized to a complimentary sequence along the single stranded amplicons. The method 350 can continue by capturing the ligated probes comprising the wild type probe and the common probe in a first region and capturing the ligated probes comprising the mutant probe and the common probe in a second region (process steps 358, 360). The method 350 can further include displaying the captured ligated probes (process step 362).

### Selected Workflows of Amplification and Ligation for Pathogen Load and Drug Resistance Testing

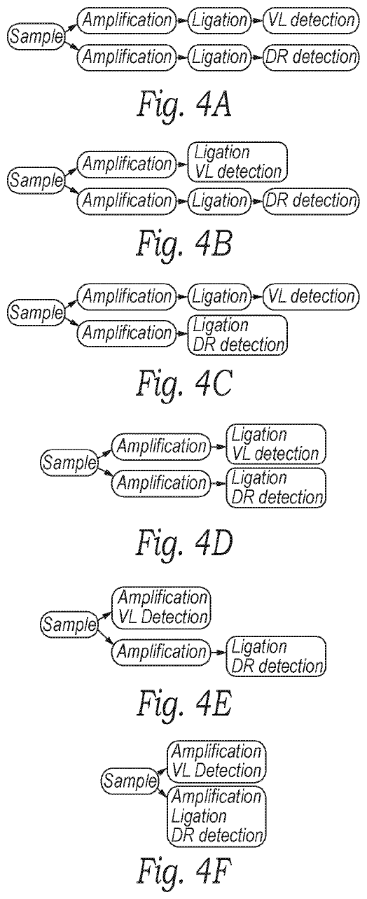

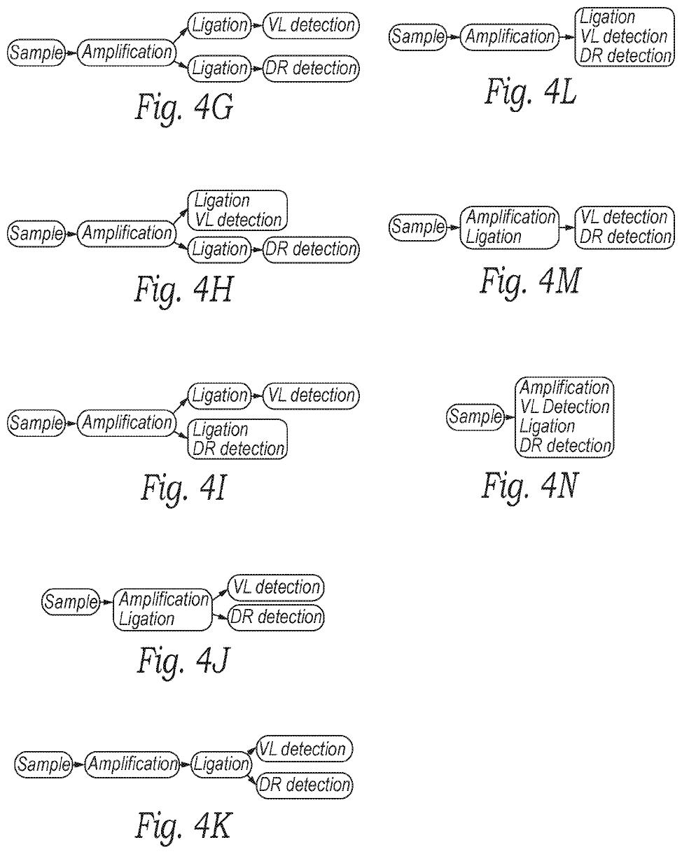

FIGS. 4A-4N are block diagrams illustrating select orders of operations to determine the pathogen load and drug resistance in a biological sample containing DNA or RNA of interest. Each block in FIGS. 4A-4N represents a physical location where the noted step is performed on a viral load and drug detection device, as will be described in greater detail below with respect to FIGS. 5A-9F. The viral load and drug detection can have a number of zones for various steps, including amplification, ligation, viral load detection, and drug resistance detection. For example, in FIG. 4A, drug resistance detection and viral load detection are in two separate blocks, indicating that these two steps are performed in physically separate regions along a viral load and drug detection device. In contrast, FIG. 4M illustrates drug resistance detection and viral load detection in the same block, which indicates these two steps are performed in the same physical region along a viral load and drug detection device. As illustrated, the steps of amplification and ligation can be combined or separated in any number of workflows. As one skilled in the art will appreciate from the disclosure herein, workflows are not limited to those expressly disclosed herein, and instead can take any form suitable for use with the technology provided herein.

### Selected OLA Devices

The present technology also provides devices architectures, systems, and devices for facilitating template-dependent ligation. For example, certain embodiments of the present technology provide disposable oligonucleotide ligation assay (OLA) devices. The OLA devices can be capable of performing the OLA workflows described herein and can be used to diagnose and characterize various infections. For example, the OLA devices described herein can be used to diagnose HIV and to determine whether an infected patient has drug resistant HIV. The OLA devices can also provide information for determining a patient's adherence to a therapeutic regimen. As will be described in detail with respect to FIGS. 5A-9B, the OLA devices can incorporate the pathogen load and drug resistance detection steps onto a lateral flow membrane. For example, an autonomous three-dimensional paper network can be used to control the release of fluids at a precise time to activate and deactivate solid-phase amplification and ligation, and the devices can contain fluid release ports that can be connected to the paper network. In some embodiments, the OLA devices can be designed for point-of-care settings to enable rapid and accurate infection diagnosis and characterization. In some embodiments, the OLA devices are designed to perform the OLA under substantially isothermal conditions.

FIG. 5A illustrates a point of care OLA device (the “device 500”) for testing the pathogen load and drug resistance of a biological sample containing genetic information. The device 500 includes a casing 502 enclosing a paper flow membrane (not shown). The casing 502 can increase the safety for medical staff conducting the test and/or reduce contamination of the device by fluidly sealing the biological sample from the external environment. The device 500 can further include a sample port 504, a buffer port 506, a pathogen load detection zone 508, and a drug resistance detection zone 510.

The sample port 504 can receive a container holding the biological sample (e.g., collection tubes). In some embodiments, the sample port 504 mates with an external surface of the container such that a bottom portion of the container is inside the casing 502 and fluidly isolated from the external environment. By fluidly isolating the bottom portion of the container from the external environment, the sample port 504 can reduce contamination from the device 500. For example, in some embodiments, the biological sample is amplified before delivering the sample to the device. In such embodiments, the sample port reduces potential contamination. To facilitate transfer between the container and the interior of the device 500, the sample port 504 can include a piercing element (not shown). The piercing element can be any element (e.g., a pin, a blade, etc.) suitable for piercing the bottom portion of the container holding the biological sample to release the biological sample onto the paper flow membrane. In other embodiments, the sample port 504 does not include a piercing element, and the biological sample is pipetted or otherwise transferred to the sample port 504.

The buffer port 506 is positioned adjacent a dried reagent pad (not shown) contained within the casing 502. The buffer port 506 is in fluid connection with the dried reagent pad such that, when liquid is added to the buffer port 506, the dried reagent pad becomes rehydrated. As will be discussed in greater detail with respect to FIGS. 8A-F, rehydrating the dried reagent pad can cause the rehydrated reagents to flow through the paper flow membrane, thus catalyzing various reactions (e.g., amplification, ligation, etc.). The buffer port 506 can include a plurality of channels 506a-e that direct the buffer to a plurality of dried reagents corresponding to the plurality of channels 506a-e. In some embodiments, the buffer can be independently added to independent receptacles corresponding to independent channels 506a-e to control the timing of rehydrating of the plurality of dried reagents. In other embodiments, the buffer is added to a single port, and the single port directs the added buffer to the plurality of channels 506a-e.

The pathogen load detection zone 508 signals the presence of a target pathogen (e.g., HIV) in the biological sample via, for example, color bands as described above with respect to FIG. 2C. The pathogen load detection zone 508 can include a plurality of regions. For example, a first region 508a can be a control region to ensure the device 500 is operating properly. A second region can indicate whether the target pathogen is present in the biological sample above a predetermined threshold (e.g., virologic failure). A third region can be a positive control that detects infection of the pathogen.

The drug resistance detection zone 510 signals whether the biological sample contains one or more sequences indicating resistance. The drug resistance detection zone 510 can be divided into a plurality of regions, with each region corresponding to a specific mutant. For example, if the device 500 is testing a biological sample for drug resistant HIV, the drug resistance detection zone 510 may have a plurality of regions 510a-f, with each region signaling the presence (or absence) of a given mutant. For example, the regions may signal the presence of the following mutants: K65R, K103N, V106M, Y181C, M184V, and G190A.

FIGS. 5B and 5C are enlarged vie % s of the pathogen load detection zone 508 and the drug resistance detection zone 510, respectively. As illustrated in FIG. 5B, the pathogen load detection zone 508 can be divided into a first region 508a, a second region 508b, and a third region 508c. As discussed previously, each region 508a-c corresponds to a specific outcome (e.g., the first region 508a indicates the device 500 is operating properly, the second region 508b indicates whether the target pathogen is present in the biological sample above a predetermined threshold, and the third region 508c indicates whether the biological sample is infected with the pathogen). The presence of the specific outcome is reported as a contrasting signal (e.g., via fluorescence or other reporter molecules). For example, the lines in FIG. 5B indicate a “positive” result for the associated indication. Each outcome reported by regions 508a-c can be interpreted together to provide patient specific information about the status of their infection. For example, a “positive” result at region 508a and 508c indicates the patient is infected and that treatment is working (i.e., the patient is infected but the target pathogen is below the predetermined threshold), a “positive” result at region 508a, 508b, and 508c indicates that the patient is infected and that treatment failed (i.e., the patient is infected and the target pathogen is above the predetermined threshold), and a “positive” result only at region 508a indicates the patient was misdiagnosed as infected.

As illustrated in FIG. 5C, the drug resistance detection zone can be divided into a plurality of regions 510a-f. Each region 510a-f can correspond to a different mutant in the pathogen conferring drug resistance to a different therapeutic regimen (e.g., FTC, TDF, EFV, NVP, AZT, 3TC). As one skilled in the at will appreciate, the drug resistance detection zone could be divided into any number of regions, and thus could test for any number of potential mutants conferring resistance. For example, in some embodiments, the drug resistance detection zone is divided into one, two, three, four, five, six, seven, eight, nine, ten, eleven, or twelve regions, and thus can test for one, two, three, four, five, six, seven, eight, nine, ten, eleven, or twelve different potential mutants conferring resistance. The presence of the specific mutant associated with a given region is reported as a contrasting signal (e.g., via fluorescence or other reporter molecule). For example, the lines in FIG. 5C indicate a “positive” result for the associated mutant.

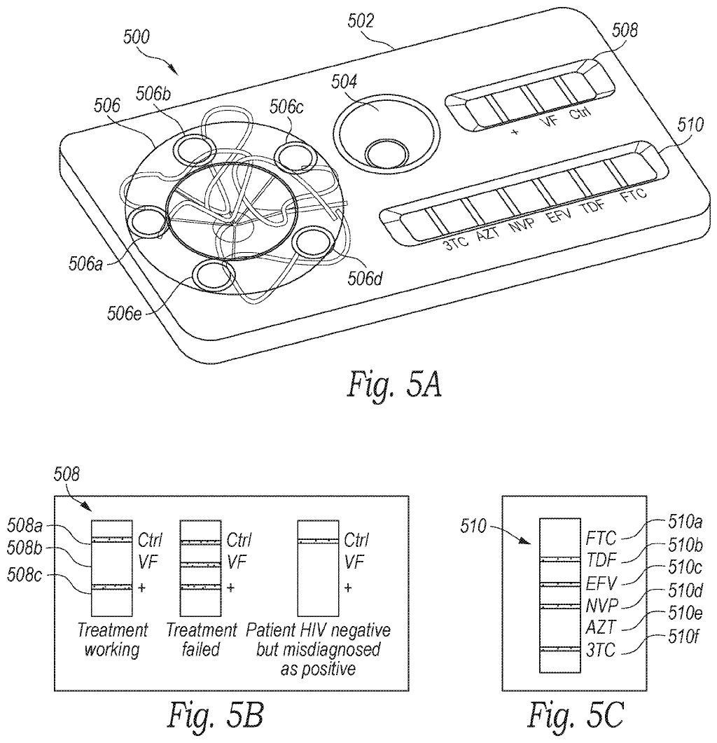

FIG. 6A is an exploded isometric view of the device 500. The casing 502 can include a top portion 502a and a bottom portion 502b. In some embodiments, the top portion 502a and the bottom portion 502b can be two separate components that releasable mate to form the casing 502. Positioned within the casing 502 is a reagent pad 512, a lateral flow membrane 520, and an absorbent pad 516.

FIG. 6B illustrates the reagent pad 512, the lateral flow membrane 520, and the absorbent pad 516. The lateral flow membrane 520 can have a length extending between the reagent pad 512 and the absorbent pad 516. The reagent pad 512 can be fluidly coupled to the lateral flow membrane 520, and the lateral flow membrane can be fluidly coupled to the absorbent pad 516. Accordingly, fluid can flow between the reagent pad 512, the lateral flow membrane 520, and the absorbent pad 516. The absorbent pad 516 (e.g., a cellulose absorbent pad) can provide a source of negative pressure to passively drive fluid flow through the lateral flow membrane 520 by creating a capillary pressure difference in the membrane. Accordingly, fluid can flow generally in the direction from the reagent pad 512 towards the absorbent pad 516. Thus, towards the reagent pad 512 can be considered an “upstream” direction, and towards the absorbent pad 516 can be considered a “downstream” direction.

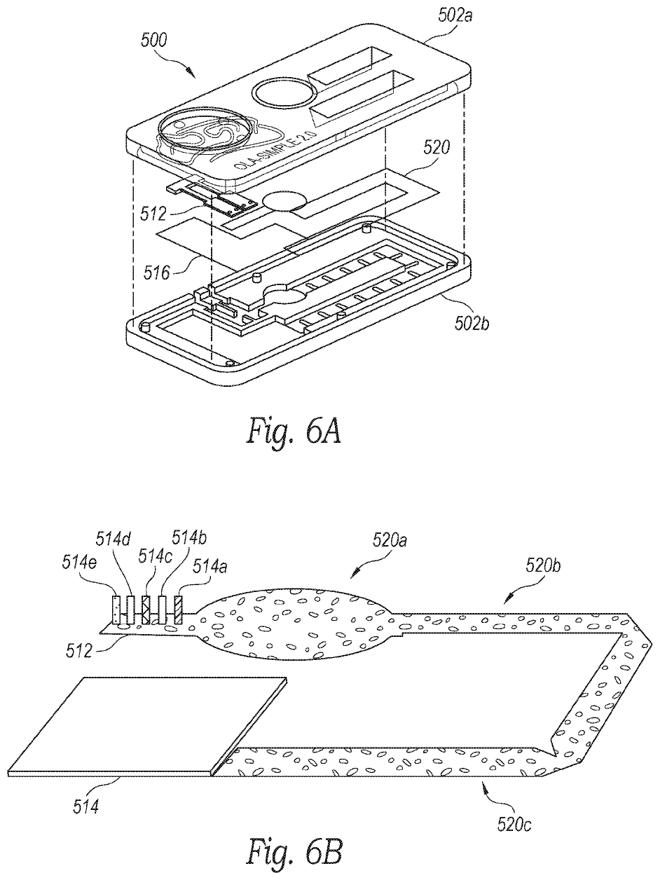

The reagent pad 512 can comprise a plurality of dried reagents 514a-e (e.g., lyophilized reagents). For example, the plurality of dried reagents 514a-e can comprise dried reagents beads prepared using the systems and methods disclosed herein with respect to FIGS. 5A-D, however dried reagents prepared using other methods may also be provided. The reagent pad 512 can be fluidly coupled to the lateral flow membrane 520 such that when the dried reagents 514a-e are rehydrated, the reagents 514a-e flow through the lateral flow membrane. Moreover, the dried reagents can be automatically and/or sequentially delivered. For example, when buffer is added to the buffer port 506 filling the plurality of channels 506a-e, the plurality of dried reagents 514a-e can be rehydrated. However, the first rehydrated reagent 514a can substantially drain (e.g., flow into the lateral flow membrane) before the second rehydrated reagent 514b can drain and flow into the lateral flow membrane. Likewise, the third rehydrated reagent 514c cannot drain and flow into the lateral flow membrane until the second rehydrated reagent 514b is substantially drained, the fourth rehydrated reagent 514d cannot drain and flow into the lateral flow membrane until the third rehydrated reagent 514c is substantially drained, and the fifth rehydrated reagent 514e cannot drain and flow into the lateral flow membrane until the fourth rehydrated reagent 514d is substantially drained. In this way, the delivery of the plurality of dried reagents 514a-e to the lateral flow membrane can be automated and sequential. In some embodiments, a user can provide an input to selectively control the timing and/or sequence of reagent delivery. For example, as described in more detail below with respect to FIGS. 7A-H, a plurality of tabs can be provided to allow user input to selectively control the delivery of reagents.

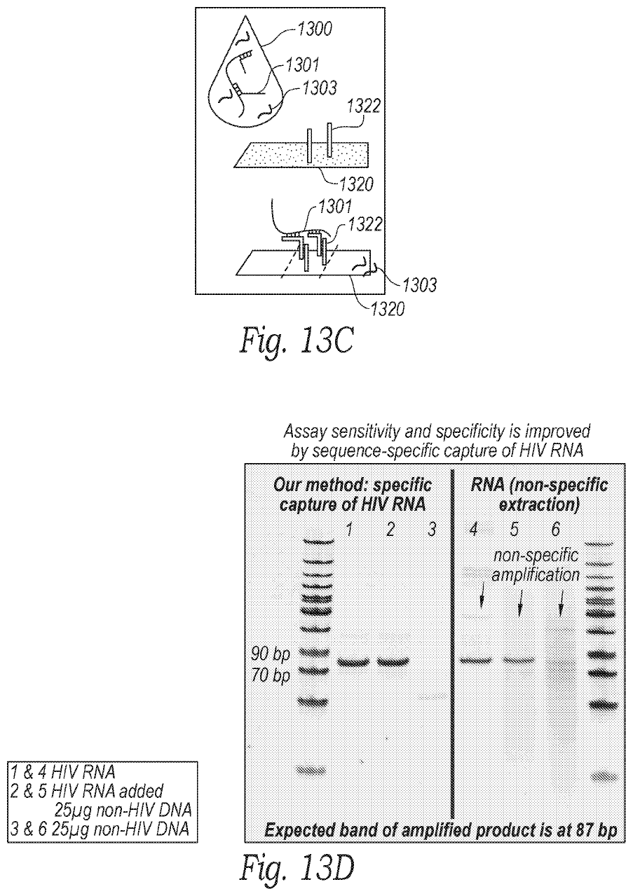

The lateral flow membrane can be divided into a first zone 520a, a second zone 520b, and a third zone 520c. The first zone 520a can receive the biological sample from the sample port 504. As described in greater detail below with respect to FIGS. 14A-C, the first zone 520a can include capture molecules for capturing and retaining a target RNA or DNA strand. For example, the first zone 520a can be embedded with probers and/or primers having nucleic acid sequences substantially complementary to a portion of the target strand. The first zone 520a can also facilitate amplification of the retained target strand. For example, the first zone 520a can include immobilized primers according to at least one of SEQ ID Nos. 41-46 to facilitate capture and amplification of the target strand. The second zone 520b can capture and retain the template strand for template dependent ligation. In some embodiments, the second zone 520b and the pathogen load detection zone 508 substantially overlap and/or are the same. The third zone 520c can capture and retain ligated probes for detecting drug resistance. Thus, the third zone 520c and the drug resistance detection zone 510 can substantially overlap and/or be the same.

FIGS. 7A-H illustrate an embodiment of the device 500 that enables a user to selectively control delivery of the dried reagents. By selectively controlling delivery of the dried reagents, the user can control the timing of the amplification and ligation steps to optimize the test results. For example, the selectively controlled delivery can delay reagent delivery to account for two incubation periods (e.g., an about 20-minute amplification step and an about 5-minute ligation period). For example, FIG. 7A illustrates the device 500 including a first pull tab 518a and a second pull tab 518b both transitionable between a closed configuration and an open configuration. FIG. 7B illustrates initial loading of the biological sample including plasma 540 and target DNA or RNA into the sample port 504 and initial rehydration of the dried reagents 514a-e. The target strand will be selectively retained in the first zone 520a, while the plasma 540 will flow through the lateral flow membrane 520 towards the absorbent pad 516, as illustrated in FIG. 7C. Once the lateral flow membrane 520 is fully wet, the first reagent 514a will be released into the lateral flow membrane 520, as illustrated in FIG. 7D. The first reagent 514a can be an amplification buffer to facilitate amplification of the retained target DNA or RNA in the first zone. The first tab 518a can prevent the second reagent 514b from flowing into the lateral flow membrane 520 even after the first reagent 514a is substantially drained. Thus, the first tab 518a can remain in a closed position to allow for the amplification incubation period. After the amplification incubation period is over, the first tab 518a can be pulled to the open position, which can allow the second reagent 514b to drain into the lateral flow membrane 520, as illustrated in FIG. 7E. The second reagent 514b can be a washing buffer. Once the washing buffer is substantially drained onto the lateral flow membrane, the third reagent 514c can be automatically drained onto the lateral flow membrane 520 to facilitate ligation in the second zone 520b, as illustrated in FIG. 7F. The second tab 518b can prevent the fourth reagent 514d from flowing into the lateral flow membrane 520 even after the third reagent 514c is substantially drained. Thus, the second tab 518b can remain in a closed position to allow for the ligation incubation period. After the ligation period is over, the second tab 518b can be pulled to the open position, which can allow the fourth reagent 514d to drain into the lateral flow membrane 520, as illustrated in FIG. 7G. The fourth reagent 514d can contain a wash that includes labeling molecules that push the ligated products downstream. Once the fourth reagent 514d is substantially drained from the reagent pad, the fifth reagent 514e can automatically drain into the lateral flow membrane 520, as illustrated in FIG. 7H. The fifth reagent 514e can include another wash fluid that contains a substrate (e.g., an enzyme or other detection molecule) that will create the color for detection. As one of skill in the art will appreciate, the device 500 can be fully automated such that a reagent release system automatically provides the amplification and ligation incubation periods without requiring user input (e.g., without requiring the user to pull the first tab 518a and the second tab 518b).

FIGS. 8A-F illustrate an exemplary workflow of the device 500 to detect a pathogen load and drug resistance of a biological sample FIG. 8A illustrates the reagent pad 512 containing the plurality of dried reagents 514a-e in a rehydrated state. FIG. 8A also illustrates the first zone 520a (e.g., the amplification zone) of the lateral flow membrane 520. As illustrated, a plurality of capture probes 522 can be immobilized in the first zone 520a of the lateral flow membrane. The capture probes 522 can include nucleotide sequences that are substantially complementary to a nucleotide sequence of a target strand 801. FIG. 8A further illustrates loading a biological sample 800 containing the target strands 801 and additional biological materials 803. As illustrated, the capture probes 522 can capture and retain the target strands 801, while permitting the additional biological materials 803 to flow through the lateral flow membrane 520 in a downstream direction. In some embodiments, the step of capturing and retaining the target strands 801 is about 10 minutes.

FIG. 8B illustrates asymmetrical amplification of the retained target strands 801. To initiate amplification, the first reagent 514a can be released from the reagent pad 512 to flow into the first zone 520a. The first reagent 514a can be an amplification buffer comprising reverse primer 524a, forward primer 524b, and polymerase (not shown). To facilitate asymmetrical amplification, the reverse primer 524a can be provided in a higher quantity and/or concentration than the forward primer 524b. Alternatively, the forward primer 524b can be provided in a higher quantity and/or concentration than the reverse primer 524a. As the target strand 801 is amplified, some of the amplicons will rehybridize to form double stranded amplicons 805 (comprising the template strand 805a and the non-template strand 805b). However, the template strand 805a will be at least partially present in single stranded form due to the excess reverse primer. As amplification continues, the polymerase will displace the strand and allow double stranded amplicons 805 and the single stranded template strands 805a to flow downstream. In some embodiments, the reverse primer can be biotinylated, such that the single-stranded template strand 808a is biotinylated. The template strand 805a can comprise cDNA substantially complimentary to the target DNA or RNA strand. In some embodiments, the template strand 805a can comprise the template strand for use with template dependent ligation. This process can continue for the duration of the amplification incubation period (e.g., about 20 minutes). After the amplification incubation period, the reagent pad 512 can release the second reagent 514b. The second reagent 514b can comprise a wash buffer that further flushes the amplicons downstream and towards the second zone 520b.

FIG. 8C illustrates the reagent pad 512 and the second zone 520b of the lateral flow membrane 520. The second zone 520b (e.g., the ligation zone) can include capture molecules 526 immobilized on the lateral flow membrane 520. For example, the capture molecules 526 can be streptavidin, and thus can capture and retain the biotinylated single stranded template strand 805a. The reagent pad 512 can release the third reagent 514c, which can comprise a ligation buffer including test probes and common probes as described herein. The ligation buffer can also contain enzymes and other materials to promote ligation (e.g. Taq DNA ligase). The test probes 528 can include a first region 529a having a sequence complimentary to a potential sequence of the single stranded amplicon 805a (and thus corresponding to a segment of the target strand 801). For example, the first test probes can have a first region comprising a first potential nucleotide sequence of a segment of the target strand (e.g., a wild type sequence), the second test probes can have a second region comprising a second potential sequence of the segment of the target strand (e.g., a first mutant sequence), the third test probes can have a third region comprising a third potential sequence of the target strand (e.g., a second mutant sequence), etc. The first test probes, second test probes, third test probes, etc. can differ from one another by a single nucleotide such that they can detect a single nucleotide polymorphism in the template strand 805a.

The test probes 528 can also include a second region 529b having a unique nucleotide sequence. For example, the first test probes can include a second region having a first unique nucleotide sequence, the second test probes can include a second region having a second unique nucleotide sequence, and the third test probes can include a second region having a third unique nucleotide sequence. The first unique nucleotide sequence, the second unique nucleotide sequence, and the third unique nucleotide sequences can be DNA barcodes that enable the first test probes, the second test probes, and the third test probes to be captured in different regions in the third zone 520c. The DNA barcodes can be computationally-designed and thus show high sensitivity (about 100% capture), low cross-reactivity, and tolerance to temperature variation from about 25-4 degrees Celsius. The barcodes can include DNA or DNA derivatives such as PNA, LNA, and/or pDNA.

The test probes 528 and/or the common probes 530 can hybridize to complementary regions along the retained template strands 805a. Ligase (e.g., Taq DNA Ligase) can ligate individual test probes 528 to individual common probes 530 when the individual test probe 528 and the individual common probe 530 are hybridized to adjacent regions of the template strand 805a to form ligated probes 531. As described herein, test probes 528 will only be ligated to common probes 530 when the test probes 528 are complementary to the region of the template strand 805a the 5′ end region of the test probe 528 is hybridized to. This process can continue for the duration of the ligation incubation period (e.g., about 5 minutes). Following the ligation incubation period, the device 500 can be heated to temperatures of about 70 degrees Celsius to denature the ligated probes and release them from the amplicons. In other embodiments, the device 500 can be heated to temperatures of about 70 degrees Celsius or higher (e.g., about 10 degrees Celsius higher than the melting temperature of the probes) and/or a wash buffer (e.g., an alkaline buffer) can be used to separate the ligated probes from the amplicons. In some embodiments, the probes can be released from the amplicons through competing hybridization. For example, invader strands comprising an identical or substantially identical sequence to the footprint of the ligated probes and a sticky end of about 6 nucleotides or more can be added to the lateral flow membrane. The sticky ends of the invader strands can bind to the template strands and undergo toehold mediated strand displacement, where the resultant invader strand-template strand duplexes are more thermodynamically stable than the test probes-template duplexes. This also reduces the likelihood of detecting a false positive in the drug resistance detection zone by reducing the likelihood that an unligated test probe and an unligated common probe will remain hybridized to the same template strand in the detection zone. In some embodiments, the sticky end can also hybridize to the strand containing the region complementary to the sticky end and the other region complementary to the probe itself. This enables unligated probes to be free from the target. In embodiments that utilize heat to denature the ligated probes, heat can be provided by a number of sources, including an on-board circuit and battery in the device 500 or a reusable heater with slots for the device 500. If a reusable heater is utilized, the same heater used for a VF test can provide ports for the device 500 (with switches to detect device inserted, status lights, communication with software, etc.). In embodiments that use competing hybridization, competing oligonucleotides can be provided to promote dissociation between the ligated probes and the template strands.

Following the ligation incubation period, the reagent pad 512 can release the fourth reagent 514c, as illustrated in FIG. 8D. The fourth reagent 514c can include competing oligos and labeling enzymes 532. The competing oligos can reduce a false signal from ligation facilitating the release of the ligated probes from the amplicons. The labeling enzymes 532 can bind to the common probes 530. For example, if the common probes 530 include a fluorescein molecule, the labeling enzymes 532 can comprise enzyme-common anti-fluorescein. Following the ligation incubation period, the ligated probes 531 can flow downstream into the third zone 520c. The third zone 520c can include immobilized capture molecules 533 having nucleotide sequences substantially complimentary to the unique nucleotide sequences of the second region 529b of the test probes 528. The third zone 520c can have a plurality of regions corresponding to different test probes. For example, the third zone 520c can have a first region corresponding to the first test probes, a second region corresponding to the second test probes, a third region corresponding to the third test probes, etc. The first region can have immobilized capture molecules comprising nucleotide sequences substantially complimentary to the first unique nucleotide sequence of the first test probes, the second region can have immobilized capture molecules comprising nucleotide sequences substantially complimentary to the second unique nucleotide sequence of the second test probes, and the third region can have immobilized capture molecules comprising nucleotide sequences substantially complimentary to the third unique nucleotide sequence. Thus, the first region can capture and retain the first test probes, the second region can capture and retain the second test probes, and the third region can capture and retain the third test probes. In this way, the ligated probes can be selectively retained in distinct regions along the lateral flow membrane 520 depending on their unique nucleotide sequences.

Following release of the fourth reagent 514c, the reagent pad 512 can release the fifth reagent 514d. The fifth reagent 514d can include a substrate that can interact with the common probes and cause the common probes to emit a signal (e.g., a color signal). For example, the substrate can interact with the labeling enzymes to cause the labeling enzymes to emit a signal. In other embodiments, the substrate can interact directly with the common probe and cause the common probe to emit a signal. The signals indicate a “positive” result for the given region the signal is associated with.

FIG. 8F illustrates the device 500 after completion of a workflow such as described with respect to FIGS. 8A-8E. As illustrated, the pathogen detection zone overlaps with the second zone 520b and signals the presence or absence of the pathogen. The drug resistance detection zone overlaps with the third zone 520c and signals the presence of absence of ligated probes, which indicates the presence or absence of specific sequences. For example, in FIG. 8E, two ligated probes were captured. Thus, the target strands 801 included strands having sequences corresponding to the first ligated probes and strands having sequences corresponding to the second ligated probes. For example, this could indicate the presence of two different single nucleotide polymorphisms in the target strands or could indicate the presence of wild type strands and mutant strands.

The entire workflow described about with respect to FIGS. 8A-8F can take about 120 minutes or less. For example, in some embodiments the workflow can take about 60 minutes or less. Thus, the device 500 can enable both HIV testing and HIV drug resistance testing in a single device and thus can be used in a point-of-care setting to make treatment decisions during a single patient visit.

The intensities of lines (or dots) reporting the presence of specific mutants from the OLA device described herein can be quantified and reported as a value that reflect the loads of single nucleotide polymorphisms detected. The higher the intensity of the line, the more prevalent the corresponding single nucleotide polymorphism is in the biological sample. Thus, signal intensities can be used to gain further information to characterize an individual's pathogen load. For example, the loads of one or more single nucleotide polymorphisms detected on lateral flow tests described herein can be analyzed and converted into one or more numbers classifying the intensity of the signal. The numbers can then be used by physicians and care givers to select an appropriate therapeutic regiment for the patient.

In some embodiments, a computer can analyze the signal and quantify the intensity of the signals via an algorithm. Exemplary algorithms can, for example, convert the detection results of a set of one or more SNP the signal obtained from lines or other shapes on the lateral flow tests or other substrates to one or more numbers, and/or convert the set of one or more numbers obtained from the detection zones into the predictive output such as but not limited to resistance or susceptibility to a particular or a combination of drug agents.

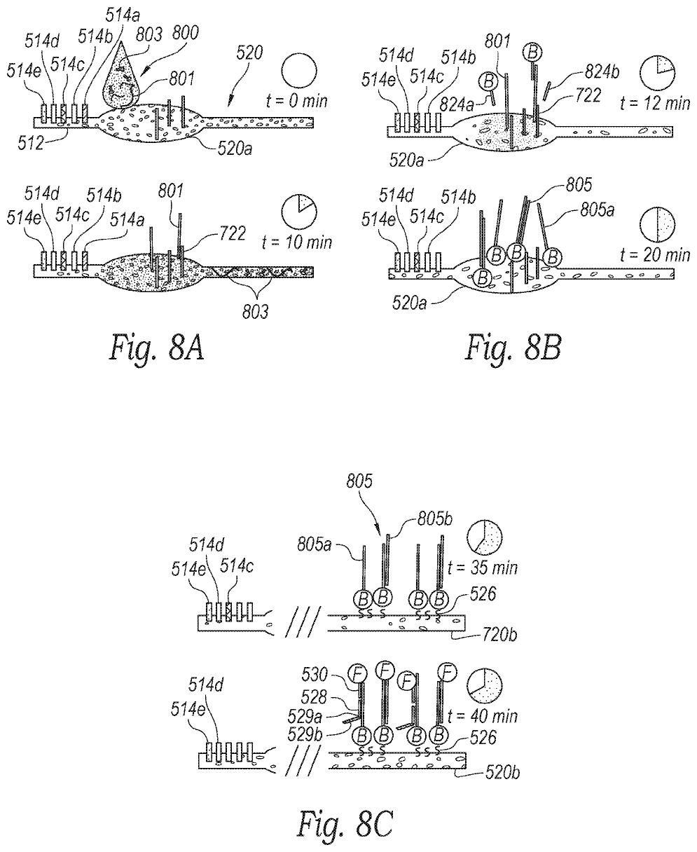

FIG. 9A illustrates another OLA device 900 in accordance with embodiments of the present technology. The device 900 can include a sample port 904, a buffer port 906 for adding rehydration buffer 907, a plurality of dried reagent pads 914a-e, and a lateral flow membrane 920. The plurality of dried reagent pads 914a-e can be separated from the lateral flow membrane 920 by a non-penetrable tab 918. The first dried reagent pad 914a can include amplification reagents. The second dried reagent pad 914b can include dissociation reagents. The third reagent pad 914c can include first ligation reagents. The fourth reagent pad 914d can include second ligation reagents. The fifth reagent pad 914e can include detection reagents. The lateral flow membrane 920 can include a target capture and reaction zone 920a having immobilized reverse primers 924 and a drug resistance detection zone 920b to display results of the drug resistance test.

A user can add a sample collection tube 934 containing a biological sample into the sample port 904. The sample port 904 can contain a piercing element that punctures the bottom of the collection tube 934 to release the biological sample onto the lateral flow membrane 920. The user can add a rehydration buffer 907 to the buffer port 906 to rehydrate the plurality of dried reagent pads 914a-e. The user can then pull the tab 918 separating the plurality of rehydrated reagents and the lateral flow membrane 920 to release the plurality of rehydrated reagents on the lateral flow membrane. As described above and further illustrated in FIG. 9B, the rehydrated reagents can be sequentially delivered to the lateral flow membrane to drive asymmetrical amplification and isothermal ligation.

As one skilled in the art will appreciate from the foregoing, a number of modifications could be made to the workflows and devices described with respect to FIGS. 5A-9B while remaining within the scope of the present technology. For example, in some embodiments, the OLA devices can be modified to test for a different number of mutations. In some embodiments, the reagents can be added sequentially rather than included within the device as dried reagents. In some embodiments, the OLA device may be designed to only perform drug resistance testing.

While the OLA devices described with respect to FIGS. 5A-9B included a lateral flow membrane, other materials and/or surfaces can be suitable for the OLA devices described herein. For example, in some embodiments the device includes other porous materials such as glass fiber, PVDF membrane, nylon membrane, and the like. In some embodiments, the device includes a two-dimensional surface such as PDMS microchips or polystyrene well plates. In some embodiments, the device may include a surface comprising microsphere beads, magnetic beads, and/or glass beads.

### Sample Collection and Preparation Devices

The present technology provides systems, devices, and associated methods for collecting and preparing a biological sample for use with systems and methods described herein. For example, before the amplification and ligation methods described herein can be performed, a biological sample containing target RNA or DNA must be obtained from an infected patient. The biological sample must also be treated such that the target RNA or DNA is available for amplification and template dependent ligation (e.g., lysed). Thus, the present technology provides an integrated sample collector and preparation device.

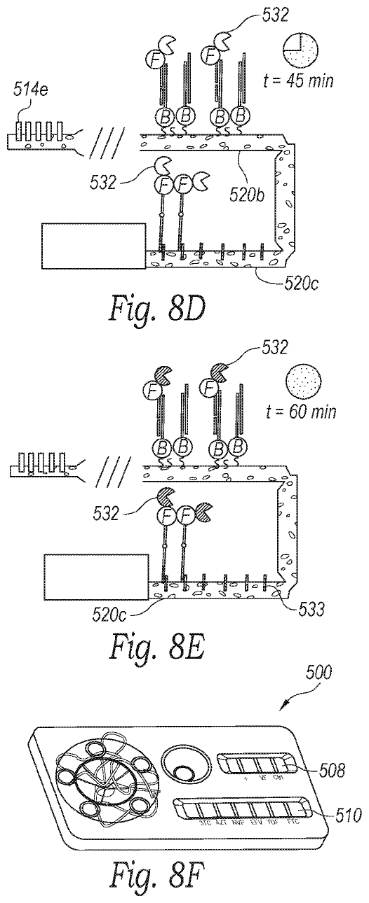

FIG. 10A illustrates an integrated sample collector and preparation device 1000 (e.g., a syringe) having a tip 1001, and collecting tube 1002, and a filter 1003. The filter 1003 can be positioned between the tip 1001 and the collecting tube 1002 such that a biological sample flowing from the tip 1001 to the collecting tube 1002 will pass through the filter 1003. The filter 1003 can be, for example, a blood filter membrane layer that filters out blood cells. The filter 1003 can allow plasma containing pathogen particles 1010 (e.g., HIV viral particles or other nucleic acids) to pass through to the collecting column. The integrated sample collector and preparation device 1000 can include a check valve (not shown) between the tip 1001 and the filter 1003 to force one-way flow of the obtained biological sample. Thus, in a first configuration for example, the integrated sample collector and preparation device 1000 could be “pulled” to draw a sample, but not “pushed” to eject a sample. In some embodiments, the collecting tube 1002 can include pre-dried lysis buffer to further prepare the biological sample.

FIG. 10B illustrates the integrated sample collector and preparation device 1000 during sample collection. The tip 1001 can be inserted into biological tissue (e.g., a human vein or tissue contained in a test tube) and the integrated sample collector and preparation device 1000 can draw the biological sample into the collecting tube 1002. The filtered plasma entering the collecting tube 1002 can rehydrate the pre-dried lysis buffer, which can then release the target pathogen genetic information (e.g., HIV RNA). The lysis buffer can also include RNAse inhibitors to prevent RNA degradation. As illustrated in FIG. 10C, the collecting tube 1002 can be used to transfer the prepared biological sample containing target RNA or DNA to OLA devices, such as those described in detail with respect to FIGS. 5A-9E.

The integrated sample collector and preparation devices provide numerous advantages over traditional sample collection and preparation devices and methods. For example, the integrated sample collector and preparation device 1000 can streamline sample collection and preparation to increase efficiency—a typical workflow of sample collection and preparation using the syringe can take about 2 minutes or less. In addition, the integrated sample collector and preparation device 1000 can be used by personal with little to no user training. The user will simply be collecting blood usual. Moreover, users will handle lysed plasma, which is less infectious than whole blood. As one skilled in the art will appreciate, the integrated sample collector and preparation device can be altered for use with various sample collection techniques. For example, the integrated sample collector and preparation device can be used with various methods for collecting a blood sample, including venipuncture and/or finger sticks.

### Sample Collection Tubes

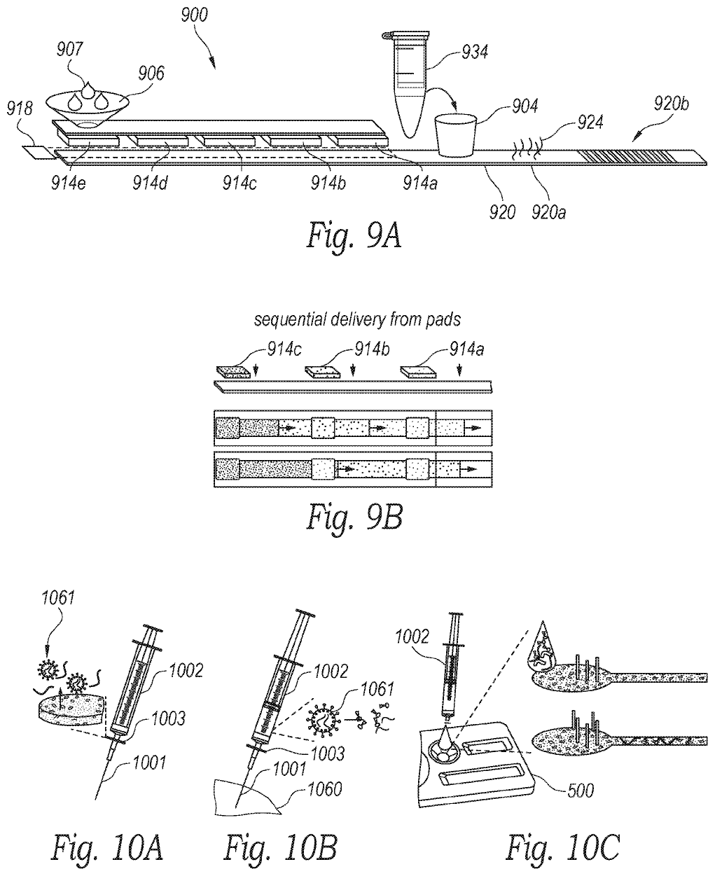

Select embodiments of the present technology provide sample collecting tubes for transferring a biological sample from a sample collection device (e.g., the integrated sample collector and preparation device 1000) to an OLA device (e.g., the devices described herein with respect to FIGS. 5A-9E). FIG. 11 illustrates an embodiment of a sample collecting tube 1100 in accordance with embodiments of the present technology. As illustrated, the sample collecting tube 1100 can include a container 1102 for housing a biological sample and a cap 1103 for fluidly sealing the interior of the container 1102 from the external environment. The cap 1103 can be transitionable between an open position and a closed position. In the open position, the interior of the container 1102 is in fluid connection with the external environment to facilitate loading of a biological sample into the collecting tube 1100. In the closed position, the interior of the container 1102 is fluidly sealed from the external environment.

The container 1102 can include a bottom portion 1106 for delivering the biological sample to the OLA device to reduce the likelihood of contamination. For example, the bottom portion 1106 can include a puncturable material. The puncturable material can include, for example, aluminum foil, wax, or any other suitable material that provides a sealed membrane that can be punctured. When the collecting tube 1100 is placed within a sample receiving port 1104 of an OLA device, the puncturable material of the bottom portion 1106 can be punctured to release the biological sample contained within the container 1102 onto the OLA device. As discussed above with respect to FIGS. 5A-9E, the sample port can include a piercing element 1105 that can puncture the bottom portion 1106 to facilitate transfer of the biological sample from the collecting tube 1100 to the OLA device. In some embodiments, the sample port 1104 may seal to an external surface of the container 1102 to prevent contamination during transfer of the biological sample from the sample collecting tube 1100 to the OLA device. As one skilled in the art will appreciate from the disclosure herein, a variety of sample collecting tubes 1100 could be manufactured and/or modified to include a bottom portion 1106 having a puncturable material to facilitate secure delivery of a sample to a test device. Accordingly, the present technology is not limited to the sample collection tubes discussed herein.

### Capturing Target Strands on a Lateral Flow Membrane

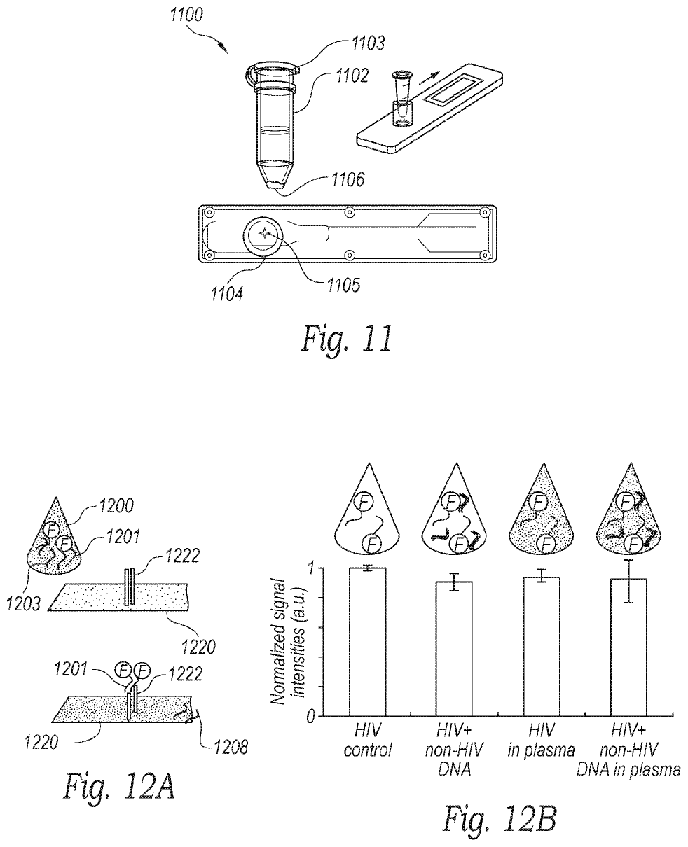

FIG. 12A illustrates a workflow for capturing target pathogens on a lateral flow membrane. Target pathogens can be captured on a lateral flow membrane using immobilized primers and/or probes that are substantially complementary to the target pathogens. As the biological sample containing the target pathogen flows through the lateral flow membrane, the target pathogens can be selectively retained in target zones along the lateral flow membrane, while non-target pathogens flow through the target zones without being retained to avoid interfering with subsequent testing (e.g., amplification and/or ligation). As illustrated in FIG. 12A, a biological sample 1200 including target pathogens 1201 (e.g., HIV RNA or DNA) and non-target biological material 1203 can be added to a lateral flow membrane 1220 having immobilized substrates 1222. The immobilized substrates 1222 can have nucleotide sequences at least partially complementary to the target pathogens 1201. Accordingly, as shown in FIG. 12A, the target pathogens 1201 will be selectively retained by hybridizing to the immobilized substrates 1222, while the non-target biological material 1203 can continue to flow through the lateral flow membrane.

FIG. 12B is a graph validating the process of selectively retaining target pathogens on a lateral flow membrane. HIV DNA was common with fluorescein (e.g., 80-bp HIV pol gene) and released onto the lateral flow membrane 1220 (e.g., nitrocellulose). HIV specific capture oligonucleotide probes were anchored on the lateral flow membrane. The presence of captured HIV was detected using a labeling enzyme and substrate that creates visible color bands (e.g., antifluorescein antibody common alkaline phosphatase/purple substrate). Four different samples were tested: (a) HIV control, (b) a combination of fluorescein common HIV and non-common non-HIV DNA, (c) fluorescein common HIV in plasma, and (d) a combination of fluorescein common HIV and non-common non-HIV in plasma. The signal intensities for the captured fluorescein common DNA for samples (b)-(d) were normalized against the control. As illustrated, the common HIV sequences were captured as they flowed through the lateral flow membrane, and the capture system was not significantly affected by the presence of human plasma and/or 25 μg of spiked non-HIV DNA.

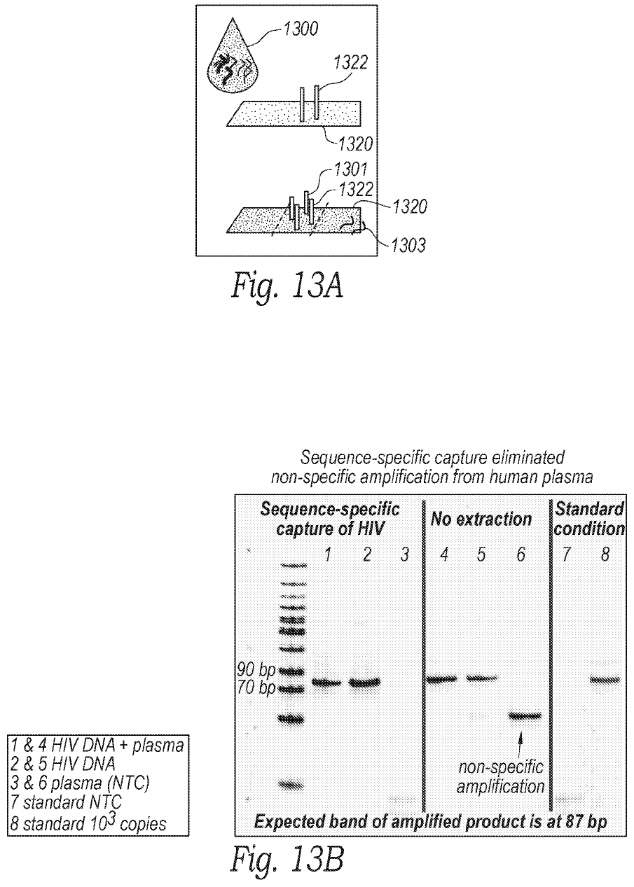

The capture method can be compatible with downstream amplification. For example, the capture method was further validated using a low (pre-amplification) level concentration of synthetic 80-base HIV pol gene (10⁴ copies). FIGS. 13A-D illustrate the results of this validation test. FIG. 13A illustrates delivering a biological sample 1300 comprising target strands 1301 and non-target material 1303 to a lateral flow membrane 1320 and capturing the target strand 1301 via immobilized capture molecules 1322. To generate the image depicted in FIG. 13B, three samples ((1) HIV DNA without plasma, (2) HIV DNA spiked in plasma, and (3) negative plasma) were wicked in paper past the capture zone containing the immobilized probes that recognize HIV. The capture zone was then excised for subsequent PCR and gel electrophoresis. Non-specific amplification is caused by the presence of non-HIV nucleic acids. The specific-capture system effectively removed non-HIV nucleic acids in plasma, as indicated by the disappearance of non-specific amplification in plasma samples (FIG. 13B, lane 3 vs. 6). A marked increase of sensitivity may occur when applying the system in lysates of HIV-infected specimens that have more cell-free nucleic acid in plasma.

For all-in-one drug resistance and viral load test. RNA in plasma is the clinically accepted specimen. The capture system described herein is also applicable for HIV RNA. FIGS. 13C and 13D illustrate the results of this validation test. FIG. 13C illustrates delivering a biological sample 1300 comprising target RNA strands 1301 and non-target maternal 1303 to a lateral flow membrane 1320 and capturing the target RNA strands 1301 via immobilized capture molecules 1322. Substituting for any suitable viral lysis buffer, the RNA model target (10⁴) is from HIV-infected plasma extracted by a commercial RNA extraction kit. The model RNA target contains both HIV and non-HIV nucleic acids that is fragmented to <10 kb from centrifugation, so it should flow through the paper membrane. To ensure efficient capturing of a whole HIV pol gene, multiple nucleotide probes were used to bind to different regions of pol. The probes have a common engineered nucleotide barcode DNA capture region that allows the RNA-probe duplex to be captured by its complementary barcode immobilized on the membrane. After the RNA-probe duplex wicked past the capture zone, the capture zone was excised for subsequent RT-PCR and gel electrophoresis. The capture method improved assay specificity and sensitivity by eliminating non-specific amplification contributed from both non-HIV RNA present in plasma (from the non-specific RNA extraction kit) and added 20 μg non-HIV DNA.

### Amplification of the Target Strand on the Lateral Flow Membrane

The present technology also provides systems and methods for amplifying the target strand on a lateral flow membrane. FIGS. 14A-14C illustrate one embodiment for selectively retaining and amplifying the target strands. FIG. 14A illustrates reverse primers 1424a immobilized on a lateral flow membrane 1420. The number of reverse primers 1424a on the lateral flow membrane 1420 can be fixed. The immobilized reverse primers 1424a can capture the target strands 1401 flowing through the lateral flow membrane 1420. Forward primers 1424b can be delivered with rehydrated amplification reagents and thus are not immobilized to the lateral flow membrane 1420. As illustrated in FIG. 14B, the reverse primers can be to create an amplicon 1405a immobilized to the lateral flow membrane 1420 that is complementary to the target strand 1401. The unwanted strands 1405b (e.g., the non-template strands) can be dissociated to provide the amplicon 1405a in single stranded form. As illustrated in FIG. 14C, the amplicons 1405 can be used to facilitate template-dependent ligation between test probes 1428 and common probes 1430.

As discussed above, the number of immobilized reverse primers 1424 can be fixed to enable primer-limited amplification. Primer-limited amplification can enable quantification of a drug resistance mutant percentage. Capturing the target strand for amplification on immobilized reverse primers also enables incompatible steps to be performed sequentially on the same device since fluid is washed away between reaction steps (e.g., between amplification and ligation). The reactions can also be spatially separated if needed, for example, to avoid probe overlap. All non-overlapping probes may be detected in one strip and overlapping probes may be reacted sequentially or split for parallel reaction. In addition, embodiments of the device may be made for different drug regimens, (e g., a patient transitioning to TDF+3TC+DTG may need RT (K65R, M184V) and DTG (N148H/R/K, N155H, R263K) in a single test).

Reverse primers can be immobilized on the lateral flow membrane by, for example, UV crosslinking of T20 tails82 or by binding biotinylated primers to immobilized streptavidin. A typical test line on nitrocellulose95 (0.1 cm thick, 1 cm wide) can bind approximately 10\hat{ }13 streptavidin molecules (50-200 ug/cm2 for IgG95), and approximately 10\hat{ }9 molecules can be seen by eye with gold labels. Thus, immobilizing 10\hat{ }12 primers (near 10\hat{ }11/cm2) is well below the binding capacity and would generate 50×DNA required to be detected if amplification and ligation were 100% efficient and sample was 100% MUT (the aim is to detect approximately 10% MUT; this would require approximately 1% combined efficiency to give detectable product; thus, there is large excess capacity and room for inefficiencies). Amplification can be carried out according to the steps detailed with respect to FIGS. 14A-D. Chemical dissociation (0.1M NaOH), as well as competing hybridization to the unwanted forward strand by peptide nucleic acid (PNA) can be used for displacing strands.

### High Throughput Fabrication of Dried Reagents

Select embodiments of the present technology provide systems and methods for high-throughput dried reagent fabrication. In some embodiments, dried reagents fabricated via the processes described herein can be incorporated into the OLA devices described with respect to FIGS. 5A-9E. As one skilled in the art will appreciate, however, the high-throughput dried reagent fabrication systems and methods described herein can be used in a variety of settings apart from the OLA devices disclosed herein.

Current processes for fabricating dried reagents take place in an industrial setting and require expensive equipment to be used to release uniformly-sized droplets containing reagent materials into liquid nitrogen for rapid freezing. For small-scale production or situations in which the expensive equipment is not accessible, technicians are required to stand for several hours to produce a large quantity (e.g., one hundred) reagent beads made by releasing the reagents from pipette tips into liquid nitrogen in a drop-wise manner. There are number of challenges associated with this approach, including: (1) the fluid will burst into small droplets if the droplets are released too fast, and (2) the fluid in the tips will freeze if the droplets are released too slowly.

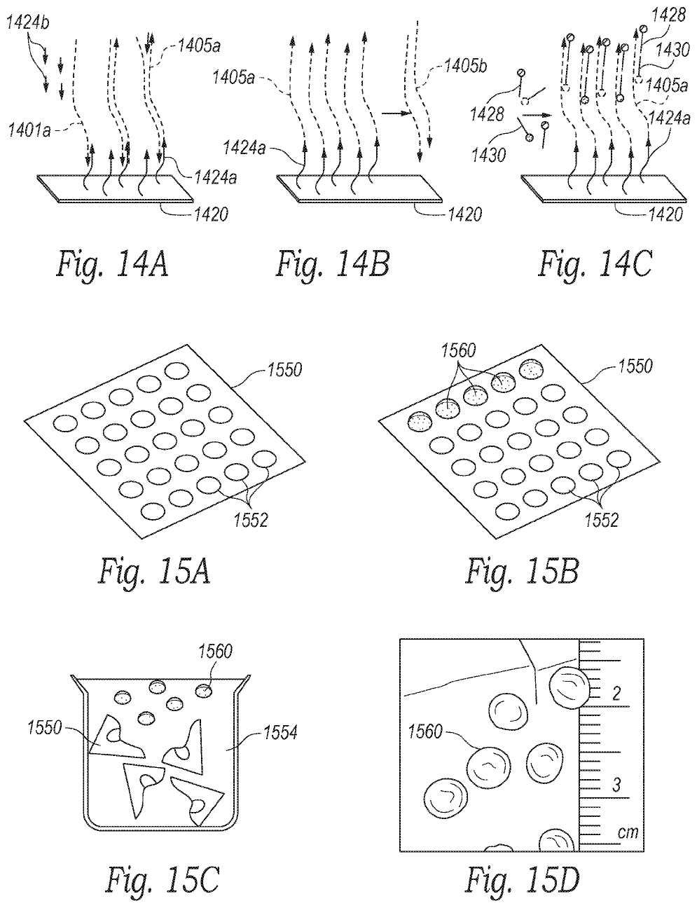

The present technology provides systems and methods for making dried reagent beads. FIG. 15A illustrates a malleable material substrate 1550 having a plurality of miniature reagent reservoirs 1552 in accordance with embodiments of the present technology. The malleable material substrate 1550 may be a wax sheet (e.g., a paraffin wax, Parafilm wax, etc.) or other suitable malleable material that is at least semi-flexible and that can retain a shape following formation of the reservoirs. For example, other materials that would either dissolve, sublimate, or crack to temperature or pressure change are possible as would be apparent to one skilled in the art. The reservoirs 1552 can be formed by pressing the malleable material over a mold (not shown) to create a plurality of indentations that define the reservoirs 1552. For example, the mold can be any plastic or other suitable material with a plurality of projections (e.g., a pipette tip rack) ne mold can be selected based off desired properties of the dried reagent beads (e.g., size of the beads, shape of the beads, etc.).

FIG. 15B illustrates droplets of reagents 1560 in the reservoirs 1552. The droplets of reagents 1560 can include preservatives, or preservatives can be added to the reservoirs 1552 in a separate step. As one skilled in the art will appreciate, any number of reagents can be added to the reservoirs 1552 to achieve a desired dried reagent bead. For example, some embodiments provide dried reagent beads 1560 with a single reagent, while other embodiments provide dried reagent beads 1560 with combinations of reagents. The dried reagents 1560 can be specifically designed to include a mixture of reagents for a particular purpose. For example, certain dried reagents 1560 may comprise reagents that facilitate asymmetrical amplification as described herein. Amplification mixtures such as RPA, HDA, and others may exclude magnesium, which can be added via a rehydration buffer or dried in a separate pad layered with the RPA mix. Other dried reagents 1560 may comprise reagents necessary to facilitate isothermal ligation as described herein. For example, ligation mixtures may include probes, ligase, buffer, and/or NAD+.

Once the reagents and/or preservatives are loaded into the reservoirs 1552, the malleable material substrate 1550 can be immersed into liquid nitrogen 1554 to cause rapid freezing of the reagents/preservatives. In other embodiments, the malleable material substrate 1550 and reagents 1560 are frozen via exposure to dry ice. The malleable material substrate 1550 can fracture when submerged into the liquid nitrogen 1554. For example, FIG. 15C illustrates the malleable material substrate 1550 fracturing into a plurality of pieces and releasing the frozen reagent droplets 1560. The frozen reagent beads 1560 can be retrieved after freezing for subsequent use. FIG. 15D illustrates dried reagent beads 1560 produced according to the procedure detailed with respect to FIGS. 15A-15C. As discussed above, the size of the dried reagent beads 1560 can be determined based on the size of the reservoirs 1552 created by the mold. Accordingly, the size and shape of the beads can be altered by changing the mold used to indent the malleable material substrate 1550. In FIG. 15D, for example, the dried reagent beads 1560 are substantially spherical and have a diameter of about 4 millimeters. In some embodiments, the beads may have a diameter between about 1 mm and about 10 mm. In other embodiments, the beads may have a diameter greater than 10 mm.

### OLA Kits

Select embodiments of the present technology include point of care kits for pathogen load testing and drug resistance detection. For example, an HIV viral load detection and drug resistance detection kit may include an OLA device, an integrated sample collector and preparation device, and sample collecting tubes as described herein. Such kits could be deliverable to point of care settings to provide rapid, low-cost HIV testing, and to enable efficacious treatment to HIV-infected patients.

### Examples

1. A method for detecting a mutation in a target strand of DNA or RNA, the method comprising:
amplifying the target strand to form amplicons;
annealing one or more of the amplicons to one or more of a plurality of probes, including—
   first test probe having a first nucleotide sequence, and
   a common probe having a second nucleotide sequence; and
ligating at least a portion of the plurality of probes via template dependent ligation based on the amplicons to generate ligated probes, wherein the ligated probes comprise first ligated probes including the first test probe ligated to the common probe; and
detecting the ligated probes, wherein detecting the first ligated probe indicates the target strand included the first nucleotide sequence.

2. The method of example 1 wherein ligating the portion of the plurality of probes to form the first ligated probe comprises:
hybridizing the first test probe to a first region of the amplicon complimentary to the first nucleotide sequence; and
hybridizing the common probe to a second region of the amplicon adjacent to the first nucleotide sequence and complementary to the third nucleotide sequence.

3. The method of example 2 wherein the first region and the second region are immediately adjacent.

4. The method of example 2 wherein the first region and the second region are spaced apart by one or more nucleotides.

5. The method of example 1 wherein the first nucleotide sequence corresponds to a mutant sequence of the target strand.

6. The method of example 1 wherein the first nucleotide sequence includes a single nucleotide polymorphism positioned at a first end region of the first test probe.

7. The method of example 1 wherein the plurality of probes further includes a second test probe having a second nucleotide sequence.

8. The method of example 7 wherein the ligated probes further include second ligated probes comprising the second test probe ligated to the common probe.

9. The method of example 8 wherein ligating the portion of the plurality of probes to form the second ligated probe comprises:
hybridizing the second test probe to a first region of the single stranded amplicon complimentary to the second nucleotide sequence; and
hybridizing the common probe to a second region of the single stranded amplicon adjacent to the second nucleotide sequence and complimentary to the third nucleotide sequence.

10. The method of example 7 wherein the second nucleotide sequence differs from the first nucleotide sequence by a single nucleotide polymorphism.

11. The method of example 7 wherein the first nucleotide sequence comprises a wild type nucleotide sequence and the second nucleotide sequence comprises a mutant of the wild type sequence.

12. The method of example 1 wherein the common probes each include one or more capture molecules, one or more reporter molecules, one or more fluorescent molecules, or one or more quencher molecules.

13. The method of example 1 wherein amplifying the target strand comprises asymmetrically amplifying the target strand to generate an excess of single stranded amplicons.

14. The method of example 13 wherein asymmetrically amplifying the target strand comprises providing one of either a forward primer or a reverse primer in a higher concentration during amplification.

15. The method of example 1 further comprising treating the amplicons to enable the amplicons to anneal to the plurality of probes.

16. The method of example 15 wherein treating the amplicons comprises heating the amplicons at a temperature of about 70-90 degrees Celsius to denature the amplicons.

17. The method of example 15 wherein treating the amplicons comprises providing an enzyme to digest one strand of the amplicons.

18. The method of example 15 wherein treating the amplicons comprises providing an enzyme that inserts one or more of the plurality of probes into the amplicons.

19. The method of example 15 wherein treating the amplicons comprises altering an environment containing the amplicons to promote de-hybridization, and wherein altering the environment comprises adding a strong base and/or reducing a salt content.

20. The method of example 15 wherein treating the amplicons comprises providing a competing oligonucleotide configured to bind to one strand of the amplicons.

21. The method of example 1 wherein the steps of amplifying, annealing, and ligating are performed at temperatures at or below about 70 degrees Celsius.

22. The method of example 1 wherein the steps of amplifying, annealing, and ligating are performed at temperatures between about 35 degrees Celsius and 45 degrees Celsius.

23. The method of example 1 wherein the steps of amplifying, annealing, and ligating are performed in a substantially isothermal environment.

24. A method for identifying drug-resistant HIV, comprising.
amplifying a target strand of RNA or DNA containing HIV genetic information to produce amplicons;
annealing one or more of the amplicons to one or more of a plurality of probes, the plurality of probes including (1) a wild type probe having a nucleotide sequence comprising a wild type sequence of the target strand, (2) at least one mutant probe having a nucleotide sequence comprising at least one potential mutant sequence of the target strand, the at least one potential mutant sequence including a single nucleotide polymorphism compared to the wild type sequence, and (3) at least one common probe;
ligating individual common probes to individual wild type probes and/or individual mutated probes to form a plurality of ligated probes each comprising a wild type probe and a common probe or a mutant probe and a common probe, wherein ligation occurs when a wild type probe or a mutant probe is hybridized to a complimentary sequence along the single stranded amplicons;
capturing the ligated probes comprising the wild type probe and the common probe in a first region; and
capturing the ligated probes comprising the mutated probe and the common probe in a second region;
displaying the captured ligated probes.

25. The method of example 24 wherein the wild type probe has a nucleotide sequence corresponding to one of SEQ ID NOs. 1, 5, 8, 11, 14, 17, 20, 23, 26, 29, or 32.

26. The method of example 24 wherein the mutant probe has a nucleotide sequence corresponding to one of SEQ ID NOs. 2, 6, 9, 12, 15, or 18, 21, 24, 27, 30, or 33.

27. The method of example 24 wherein amplifying the target strand comprises asymmetrically amplifying the target strand to generate an excess of single stranded amplicons.

28. The method of example 27 wherein asymmetrically amplifying the target strand comprises providing one of either a forward primer or a reverse primer in a higher concentration during amplification.

29. The method of example 24 further comprising treating the amplicons to enable the amplicons to anneal to the plurality of probes.

30. The method of example 24 wherein treating the amplicons comprises heating the amplicons at a temperature of about 70-90 degrees Celsius to denature the amplicons.

31. The method of example 24 wherein treating the amplicons comprises providing an enzyme to digest one strand of the amplicons.

32. The method of example 24 wherein treating the amplicons comprises providing an enzyme that inserts one or more of the plurality of probes into the amplicons.

33. The method of example 24 wherein treating the amplicons comprises altering an environment containing the amplicons to promote de-hybridization, and wherein altering the environment comprises adding a strong base and/or reducing a salt content.

34. The method of example 24 wherein treating the amplicons comprises providing a competing oligonucleotide configured to bind to one strand of the amplicons.

35. The method of example 24 wherein the steps of amplifying, annealing, and ligating are performed at temperatures at or below about 70 degrees Celsius.

36. The method of example 24 wherein the steps of amplifying, annealing, and ligating are performed at temperatures between about 35 degrees Celsius and 45 degrees Celsius.

37. The method of example 24 wherein the steps of amplifying, annealing, and ligating are performed in a substantially isothermal environment.

38. A device for identifying mutants in a target strand of DNA or RNA, the device comprising:
a reagent pad including a plurality of dried reagents;
a sample port configured to receive a biological sample including the target strand of DNA or RNA; and
a lateral flow membrane fluidly coupled to the sample port and the reagent pad and configured to receive the biological sample and the plurality of dried reagents flowing from the reagent pad after rehydration, wherein the lateral flow membrane comprises—
   an amplification zone configured to receive and capture the target strand to provide for amplification of the target strand,
   a ligation zone configured to capture amplicons of the target strand to provide for template dependent ligation, and
   a detection zone configured to capture ligated probes indicative of the presence of one or more mutants in the target strand, wherein the detection zone is configured to display a signal indicative of the presence of the captured ligated probes.

39. The device of example 38, further comprising an absorbent pad spaced apart from the dehydrated reagent pad by a length of the lateral flow membrane, wherein the absorbent pad is configured to facilitate fluid flow through the length of the lateral flow membrane.

40. The device of example 38 wherein the device is configured to automatically and sequentially deliver the plurality of dried reagents following rehydration of the reagent pad.

41. The device of example 38 wherein the plurality of dried reagents includes a first reagent for use in the amplification zone and a second reagent for use in the ligation zone, and wherein, after rehydration, the first reagent flows through the lateral flow membrane before the second reagent flows through the lateral flow membrane.

42. The device of example 41 wherein the dried reagent pad is configured such that the release of the second reagent is delayed for a period of time following the release of the first reagent to provide an amplification incubation period.

43. The device of example 42, further comprising a tab configured to control the release of the second reagent, wherein the tab is transitionable between a first position in which the second reagent is retained in the reagent pad and a second position in which the second reagent is released onto the lateral flow membrane.

44. The device of example 38 wherein the plurality of dried reagents include forward primers and reverse primers, and wherein, when the dried reagents are rehydrated, the forward primers and the reverse primers flow through a portion of the lateral flow membrane to the amplification zone.

45. The device of example 44 wherein the forward primers have a first concentration in the dried reagent and the reverse primers have a second concentration in the dried reagent, and wherein the first concentration differs from the second concentration.

46. The device of example 38 wherein the plurality of dried reagents include a plurality of probes, and wherein, when the dried reagents are rehydrated, the plurality of probes flow through a portion of the lateral flow membrane to the ligation zone.

47. The device of example 46 wherein the plurality of probes includes:
mutant probes having a nucleotide sequence comprising at least one potential mutant sequence of the target strand, the at least one potential mutant sequence including a single nucleotide polymorphism compared to a wild type sequence; and
one or more common probes.

48. The device of example 47 wherein the one or more common probes are configured to be ligated to the mutant probes in the ligation zone.

49. The device of example 47 wherein the plurality of probes further includes wild type probes having a nucleotide sequence comprising a wild type sequence of the target strand.

50. The device of example 49 wherein the one or more common probes are configured to be ligated to either the wild type probes or the mutant probes in the ligation zone.

51. The device of example 38 wherein the amplification zone includes a plurality of immobilized primers at least partially complimentary to the target strand.

52. The device of example 38 wherein the detection zone includes a plurality of regions, and wherein each region of the plurality of regions has a unique capture molecule immobilized on the lateral flow membrane.

53. The device of example 52 wherein each of the plurality of regions corresponds to a different specific sequence of the target strand, wherein the different specific sequences include one or more mutant sequences.

54. The device of example 53 wherein the sample port includes a piercing element configured to pierce a housing containing the biological sample to release the biological sample onto the lateral flow membrane.

55. The device of example 38 wherein the device is configured for isothermal amplification and/or isothermal ligation.

56. A device for identifying drug resistant HIV, the device comprising:
a dehydrated reagent pad including a plurality of dried reagents; and
a lateral flow membrane fluidly coupled to the dehydrated reagent pad and including an amplification and ligation zone and a detection zone, wherein the amplification and ligation zone is configured to—
   capture a target strand containing HIV genetic information to provide for amplification of the target strand and production of amplicons,
   receive a plurality of probes, wherein the plurality of probes includes
      mutant probes having a nucleotide sequence corresponding to at least one potential mutant sequence of the target strand, the at least one potential mutant sequence, and
      common probes,
   wherein the plurality of probes are configured to undergo template dependent ligation based on the amplicons to form a plurality of ligated probes, wherein each individual ligated probe comprises one mutant probe and one common probe;
wherein the detection zone is configured to capture the ligated probes that include the mutant probe.

57. The device of example 56 wherein the amplification and ligation zone includes a first region configured to capture the target strand and a second region configured to capture single stranded amplicons.

58. The device of example 57 wherein the first region of the amplification and ligation zone is configured to facilitate amplification of the target strand and the second region of the amplification and ligation zone is configured to facilitate ligation of the plurality of probes.

59. The device of example 56 wherein the amplification and ligation zone include a first region configured to capture the target strand, and wherein the first region is configured to facilitate amplification of the target strand and ligation of the plurality of probes.

60. The device of example 56 wherein ligation occurs when (a) the mutant probe is hybridized to a first region of the amplicon complimentary to the mutant probe, and (b) the common probe is hybridized to a second region of the amplicon complimentary to the common probe.

61. The device of example 60 wherein the first region and second region of the amplicon are immediately adjacent.

62. The device of example 60 wherein the first region and the second region of the amplicon are spaced apart one or more nucleotides.

63. The device of example 56 wherein the device is configured for isothermal amplification and ligation.

64. The device of example 56 wherein the common probes include a reporter molecule.

65. The device of example 56 wherein the mutant probes are first mutant probes corresponding to a first potential mutation, the mutant probes further comprising second mutant probes corresponding to a second potential mutation.

66. The device of example 65 wherein the plurality of ligated probes include first ligated probes comprising the first mutant probe and the common probe and/or second ligated probes comprising the second mutant probe and the common probe.

67. The device of example 66 wherein the detection zone includes a first region configured to detect the first ligated probes and a second region configured to detect the second ligated probes.

68. The device of example 67 wherein—
the first mutant probe includes a first end region configured for ligation to the common probe and a second region having a first DNA barcode,
the second mutant probe includes a first end region configured for ligation to the common probe and a second region having a second DNA barcode,
wherein the second DNA barcode is different than the first DNA barcode.

69. The device of example 68 wherein the first region of the detection zone includes immobilized capture molecules comprising a first nucleotide sequence substantially complimentary to the first DNA barcode, and wherein the second region of the third zone includes immobilized capture molecules comprising a second nucleotide sequence substantially complimentary to the second DNA barcode.

70. The device of example 67 wherein the first mutant probes include a first immuno-tag and the second mutant probes include a second immuno-tag, and wherein the first immuno-tag and the second immuno-tag are different.

71. The device of example 70 wherein the first region of the detection zone includes immobilized antibodies configured to bind the first immuno-tag, and wherein the second region of the third zone includes immobilized antibodies configured to bind the second immuno-tag.

72. The device of example 56 wherein the dried reagents include the mutant probes and the common probes.

73. The device of example 56 wherein the dried reagents include reverse primers and/or forward primers.

74. The device of example 56 wherein the amplification and ligation zone includes immobilized reverse primers and/or immobilized forward primers.

75. The device of example 56, further comprising a sample port configured to receive a biological sample including the target strand.

76. The device of example 75 wherein the sample port is fluidly coupled to the lateral flow membrane and is configured to receive a container housing the biological sample, and wherein the sample port includes a piercing element configured to pierce the container to release the biological sample onto the lateral flow membrane.

77. The device of example 75 wherein the sample port is configured to deliver the biological sample to the amplification and ligation zone.

78. The device of example 56 wherein the at least one potential mutant sequence includes a single nucleotide polymorphism compared to a wild type sequence.

79. The device of example 56 wherein the mutant probes have a nucleotide sequence corresponding to one or more of SEQ ID NOs: 2, 6, 9, 12, 15, or 18, 21, 24, 27, 30, or 33.

80. A device for in-situ isothermal amplification and ligation, the device comprising:
a dehydrated reagent pad comprising a plurality of dried reagents including one or more test probes having nucleotide sequences comprising one or more sequences corresponding to target strands of DNA or RNA;
a lateral flow membrane in fluid communication with the dehydrated reagent pad, wherein the lateral flow membrane includes:
   a capture and amplification zone configured to retain and amplify a target strand,
   a template dependent ligation zone for ligating one or more test probes, wherein the template dependent ligation zone has capture molecules immobilized on the lateral flow membrane and is configured to capture and retain an amplicon of the target strand, and
   a detection zone having a plurality of regions configured to capture ligated probes, wherein the detection zone is further configured to display a signal indicative of the captured ligated probes;
wherein the device is configured to facilitate amplification of the target strand and ligation of the one or more test probes in a substantially isothermal manner.

### CONCLUSION

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein.” “above.” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A method for detecting a mutation in a target strand of DNA or RNA, the method comprising:
amplifying the target strand to form amplicons;
annealing one or more of the amplicons to one or more of a plurality of probes, including—
a first test probe having a first nucleotide sequence, and
a common probe having a second nucleotide sequence; and
ligating at least a portion of the plurality of probes via template dependent ligation based on the amplicons to generate ligated probes, wherein the ligated probes comprise first ligated probes including the first test probe ligated to the common probe; and
detecting the ligated probes, wherein detecting the first ligated probe indicates the target strand included the first nucleotide sequence.

2. The method of claim 1 wherein ligating the portion of the plurality of probes to form the first ligated probe comprises:
hybridizing the first test probe to a first region of the amplicon complimentary to the first nucleotide sequence; and
hybridizing the common probe to a second region of the amplicon adjacent to the first nucleotide sequence and complementary to the second nucleotide sequence.

3. The method of claim 2 wherein the first region and the second region are immediately adjacent.

4. The method of claim 2 wherein the first region and the second region are spaced apart by one or more nucleotides.

5. The method of claim 1 wherein the first nucleotide sequence corresponds to a mutant sequence of the target strand.

6. The method of claim 1 wherein the first nucleotide sequence includes a single nucleotide polymorphism positioned at a first end region of the first test probe.

7-12. (canceled)

13. The method of claim 1 wherein amplifying the target strand comprises asymmetrically amplifying the target strand to generate an excess of single stranded amplicons.

14. (canceled)

15. The method of claim 1 further comprising treating the amplicons to enable the amplicons to anneal to the plurality of probes.

16. The method of claim 15 wherein treating the amplicons comprises heating the amplicons at a temperature of about 70-90 degrees Celsius to denature the amplicons.

17. The method of claim 15 wherein treating the amplicons comprises providing an enzyme to digest one strand of the amplicons.

18. The method of claim 15 wherein treating the amplicons comprises providing an enzyme that inserts one or more of the plurality of probes into the amplicons.

19. The method of claim 15 wherein treating the amplicons comprises altering an environment containing the amplicons to promote de-hybridization, and wherein altering the environment comprises adding a strong base and/or reducing a salt content.

20. The method of claim 15 wherein treating the amplicons comprises providing a competing oligonucleotide configured to bind to one strand of the amplicons.

21. The method of claim 1 wherein the steps of amplifying, annealing, and ligating are performed at temperatures at or below about 70 degrees Celsius.

22. The method of claim 1 wherein the steps of amplifying, annealing, and ligating are performed at temperatures between about 35 degrees Celsius and 45 degrees Celsius.

23. The method of claim 1 wherein the steps of amplifying, annealing, and ligating are performed in a substantially isothermal environment.

24. A method for identifying drug-resistant HIV, comprising:
amplifying a target strand of RNA or DNA containing HIV genetic information to produce amplicons;
annealing one or more of the amplicons to one or more of a plurality of probes, the plurality of probes including (1) a wild type probe having a nucleotide sequence comprising a wild type sequence of the target strand, (2) at least one mutant probe having a nucleotide sequence comprising at least one potential mutant sequence of the target strand, the at least one potential mutant sequence including a single nucleotide polymorphism compared to the wild type sequence, and (3) at least one common probe;
ligating individual common probes to individual wild type probes and/or individual mutated probes to form a plurality of ligated probes each comprising a wild type probe and a common probe or a mutant probe and a common probe, wherein ligation occurs when a wild type probe or a mutant probe is hybridized to a complimentary sequence along the amplicons;
capturing the ligated probes comprising the wild type probe and the common probe in a first region; and
capturing the ligated probes comprising the mutated probe and the common probe in a second region.

25. (canceled)

26. (canceled)

27. The method of claim 25 wherein amplifying the target strand comprises asymmetrically amplifying the target strand to generate an excess of single stranded amplicons.

28. The method of claim 27 wherein asymmetrically amplifying the target strand comprises providing one of either a forward primer or a reverse primer in a higher concentration during amplification.

29. The method of claim 24 further comprising treating the amplicons to enable the amplicons to anneal to the plurality of probes.

30-34. (canceled)

35. The method of claim 24 wherein the steps of amplifying, annealing, and ligating are performed at temperatures at or below about 70 degrees Celsius.

36. The method of claim 24 wherein the steps of amplifying, annealing, and ligating are performed at temperatures between about 35 degrees Celsius and 45 degrees Celsius.

37. The method of claim 24 wherein the steps of amplifying, annealing, and ligating are performed in a substantially isothermal environment.

38-80. (canceled)
